SEQUENCE LISTING (5 sequences)

SEQ ID NO: 1 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 2 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 3 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 4 - protein, 607 aa, Homo sapiens
MAEKRRGSPCSMLSLKAHAFSVEALIGAEKQQQLQKKRRKLGAEEAAGAVDDGGCSRGGGAGEKGSSEGDEGAALPPPAGATSGPARSGADLERGAAGGCEDGFQQGASPLASPGGSPKGSPARSLARPGTPLPSPQAPRVDLQGAELWKRFHEIGTEMIITKAGRRMFPAMRVKISGLDPHQQYYIAMDIVPVDNKRYRYVYHSSKWMVAGNADSPVPPRVYIHPDSPASGETWMRQVISFDKLKLTNNELDDQGHIILHSMHKYQPRVHVIRKDCGDDLSPIKPVPSGEGVKAFSFPETVFTTVTAYQNQQITRLKIDRNPFAKGFRDSGRNRMGLEALVESYAFWRPSLRTLTFEDIPGIPKQGNASSSTLLQGTGNGVPATHPHLLSGSSCSSPAFHLGPNTSQLCSLAPADYSACARSGLTLNRYSTSLAETYNRLTNQAGETFAPPRTPSYVGVSSSTSVNMSMGGTDGDTFSCPQTSLSMQISGMSPQLQYIMPSPSSNAFATNQTHQGSYNTFRLHSPCALYGYNFSTSPKLAASPEKIVSSQGSFLGSSPSGTMTDRQMLPPVEGVHLLSSGGQQSFFDSRTLGSLTLSSSQVSAHMV

SEQ ID NO: 5 - protein, 1367 aa, Homo sapiens
MESKALLAVALWFCVETRAASVGLPGDFLHPPKLSTQKDILTILANTTLQITCRGQRDLDWLWPNAQRDSEERVLVTECGGGDSIFCKTLTIPRVVGNDTGAYKCSYRDVDIASTVYVYVRDYRSPFIASVSDQHGIVYITENKNKTVVIPCRGSISNLNVSLCARYPEKRFVPDGNRISWDSEIGFTLPSYMISYAGMVFCEAKINDETYQSIMYIVVVVGYRIYDVILSPPHEIELSAGEKLVLNCTARTELNVGLDFTWHSPPSKSHHKKIVNRDVKPFPGTVAKMFLSTLTIESVTKSDQGEYTCVASSGRMIKRNRTFVRVHTKPFIAFGSGMKSLVEATVGSQVRIPVKYLSYPAPDIKWYRNGRPIESNYTMIVGDELTIMEVTERDAGNYTVILTNPISMEKQSHMVSLVVNVPPQIGEKALISPMDSYQYGTMQTLTCTVYANPPLHHIQWYWQLEEACSYRPGQTSPYACKEWRHVEDFQGGNKIEVTKNQYALIEGKNKTVSTLVIQAANVSALYKCEAINKAGRGERVISFHVIRGPEITVQPAAQPTEQESVSLLCTADRNTFENLTWYKLGSQATSVHMGESLTPVCKNLDALWKLNGTMFSNSTNDILIVAFQNASLQDQGDYVCSAQDKKTKKRHCLVKQLIILERMAPMITGNLENQTTTIGETIEVTCPASGNPTPHITWFKDNETLVEDSGIVLRDGNRNLTIRRVRKEDGGLYTCQACNVLGCARAETLFIIEGAQEKTNLEVIILVGTAVIAMFFWLLLVILVRTVKRANEGELKTGYLSIVMDPDELPLDERCERLPYDASKWEFPRDRLKLGKPLGRGAFGQVIEADAFGIDKTATCKTVAVKMLKEGATHSEHRALMSELKILIHIGHHLNVVNLLGACTKPGGPLMVIVEFSKFGNLSTYLRGKRNEFVPYKSKGARFRQGKDYVGELSVDLKRRLDSITSSQSSASSGFVEEKSLSDVEEEEASEELYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHEDPNQRPSFSELVEHLGNLLQANAQQDGKDYIVLPMSETLSMEEDSGLSLPTSPVSCMEEEEVCDPKFHYDNTAGISHYLQNSKRKSRPVSVKTFEDIPLEEPEVKVIPDDSQTDSGMVLASEELKTLEDRNKLSPSFGGMMPSKSRESVASEGSNQTSGYQSGYHSDDTDTTVYSSDEAGLLKMVDAAVHADSGTTLQLTSCLNGSGPVPAPPPTPGNHERGAA

(19) United States
(12) Patent Application Publication
(10) Pub. No.: US 2010/0285580 A1
(43) Pub. Date: Nov. 11, 2010

(54) Cardiac Conduction System (CCS) Progenitor Cells and Uses Thereof

(76) Inventors: Sylvia Evans, Del Mar, CA (US); Yunfu Sun, San Diego, CA (US); Ju Chen, San Diego, CA (US); Taylor Liu, Salinas, CA (US)

Correspondence Address: DLA PIPER LLP (US), 4365 EXECUTIVE DRIVE, SUITE 1100, SAN DIEGO, CA, 92121-2133, US

(21) Appl. No.: 12/742,248

(22) PCT Filed: Nov. 14, 2008
(86) PCT No.: PCT/US2008/083579

§ 371 Date: Jul. 22, 2010

Related U.S. Application Data
(60) Provisional application No. 60/988,307, filed on Nov. 15, 2007.

(51) Int. Cl. C12N 5/071

(52) U.S. Cl. 435/354; 435/325; 435/366

(57) ABSTRACT

The present invention provides isolated stem cells and progenitor cells, including HCN4-expressing CCS progenitor cells, HCN4- and Islet 1-expressing sinoatrial node cells or progenitor cells thereof, and Islet 1-expressing cardiovascular stem cells that do not express HCN4. The invention further provides differentiated cell types derived from the CCS progenitor cells and cardiovascular stem cells of the invention. In addition, the invention provides methods of differentiating the cells of the invention, methods for screening for agents that affect the differentiation of the cells of the invention, methods for evaluating the potential toxicity of drugs using the cells of the invention, cell therapy using the cells of the invention, and methods of treating heart arrhythmia by ablating HCN4-expressing cells.

### FIELD OF THE INVENTION

The invention relates generally to cardiac progenitor cells, the isolation of such cells, and their uses. More specifically, this invention relates to the isolation and use of HCN4-expressing CCS progenitor cells, HCN4- and Islet1-expressing sinoatrial node cells, and Islet1-expressing cardiovascular stem cells that do not express HCN4.

### BACKGROUND OF THE INVENTION

Cardiac Arrhythmia is the leading cause of death in adults. Arrhythmic foci and triggers of arrhythmia can be precisely mapped by programmed electrical stimulation and intracardiac recordings. However, the molecular and cellular nature of the arrhythmia is largely unknown.

Cardiac arrhythmias occur preferentially in areas derived from the developing cardiac conduction system (CCS). The CCS consists of the sinoatrial node (SAN), atrioventricular node (AVN), and peripheral components of the fast conducting His-Purkinje fibers. Although great progress has been made in understanding the anatomy and physiology of the CCS, little is known as to the developmental origins of pacemaker and conduction system cells. Lineage analysis in chick has provided evidence that ventricular Purkinje fibers are recruited from a pool of cardiomyogenic precursors that are in close contact to developing coronary arteries and subendocardium. However, the ontogenesis of pacemaker cells in nodal tissues is unknown.

Cellular automaticity and excitability in the cardiac conduction system result from activities of a diversity of ion channels. The pacemaker current (I_{f} or Iₕ) is encoded by a family of Hyperpolarization-activated, Cyclic Nucleotide gated (HCN) channels and plays a key role in the generation and autonomic regulation of sinus rhythm and rate. Four mammalian HCN isoforms (HCN1-4) have been identified, of which HCN4 is most abundantly expressed in the sinoatrial node. Previous studies have revealed early expression of HCN4 mRNA in a localized domain at cardiac crescent stages, and it specifically marks the SAN region during development and in adult. In addition, previous studies have observed, well before the coronary vascularization, the formation of a pacemaker region at the inflow tract of early heart tube.

Abnormalities in CCS development have been postulated to predispose individuals to arrhythmias and sudden death. Thus understanding of genes and signaling pathways underlying CCS development is of critical importance to gain molecular insights into human cardiac arrhythmogenesis and to develop effective interventive and regenerative therapies.

### SUMMARY OF THE INVENTION

The present invention is based, in part, on the discovery that Hyperpolarization-activated, Cyclic Nucleotide gated (HCN) channel-4 (HCN4) is expressed in all components of the cardiac conduction system (CCS), including the pacemaking centers, the sinoatrial (SA) and atrioventricular (AV) nodes, and the His-Purkinje system, during development of the conduction system and in the adult. The present invention is also based, in part, on the discovery that HCN4 is expressed in cardiomyocyte progenitor cells. The present invention is also based, in part, on the discovery that co-expression of HCN4 and Islet1 specifically identifies SA node cells and their progenitors. The present invention is also based, in part, on the discovery that HCN4 expression marks regions of the heart where focal atrial tachycardias are observed in human patients.

Accordingly, in one aspect, the invention provides isolated CCS progenitor cells that express HCN4. The CCS progenitor cells can give rise to one or more components of the CCS, such as SA node cells, AV node cells, and/or His-Purkinje fiber cells. Alternatively, the CCS progenitor cells can give rise to cardiomyocytes. The CCS progenitor cells can express one or more markers associated with stem cells or progenitor cells, including, for example, Islet1. The CCS progenitor cells can be isolated and/or derived from embryonic, postnatal, adolescent or adult tissues, including mesenchymal tissues, heart tissues, epicardial tissues, serosal mesothelial tissues, adipose tissues, dermal tissues, bone marrow, or umbilical cord. The CCS progenitor cells can be mammalian cells, such as mouse or human cells. In one embodiment, at least 75%, 80%, 85%, 90%, 95%, 98%, or more of the cells in the isolated population of CCS progenitor cells express HCN4 (i.e., are HCN4+).

In another aspect, the invention provides isolated cardiovascular stem cells that express Islet1 but not HCN4. The cardiovascular stem cells can give rise to heart tissues, including endocardium (e.g., vascular cells) and cardiac smooth muscle (e.g., cardiomyocytes). The cardiovascular stem cells can express one or more markers other than Islet1 associated with stem cells or progenitor cells, such as Nkx2.5, flk1, and/or LeX/SSEA1/CD15. The cardiovascular stem cells can be isolated and/or derived from embryonic, postnatal, adolescent or adult tissues, including mesenchymal tissues, heart tissues, epicardial tissues, serosal mesothelial tissues, adipose tissues, dermal tissues, bone marrow, or umbilical cord. The cardiovascular stem cells can be mammalian cells, such as mouse or human cells.

In another aspect, the invention provides methods of isolating progenitor cells that express HCN4. The methods include isolating cells that express HCN4 from a population of cells, such as a population of stem and/or progenitor cells. HCN4-expressing progenitor cells can be isolated, for example, by detecting HCN4 expression in the population of cells and isolating the HCN4-expressing cells. Optionally, isolation of HCN4-expressing progenitor cells can involve a stem cell and/or progenitor cell selection step, such as growing the population of cells on a feeder cell layer or detecting expression of a stem/progenitor cell marker, such as Islet1, Nkx2.5, Tbx18, flk1, or SSEAVCD15. The stem cell and/or progenitor cell selection step can precede or follow the step of isolating HCN4-expressing cells. Optionally, isolation of HCN4-expresssing progenitor cells can involve contacting the population of cells with an agent that promotes or maintains the expression of a stem/progenitor cell marker, such as Islet1, Nkx2.5, Tbx18, flk1, or SSEA1/CD15.

In another aspect, the invention provides methods of isolating sinoatrial (SA) node cells or progenitors thereof. The methods include isolating cells that co-express Islet1 and HCN4 from a population of cells, such as a population of stem and/or progenitor cells. The methods can involve, for example, detecting HCN4 expression in the population of cells and isolating the HCN4-expressing cells. The methods can further involve, for example, detecting Islet1 expression in the population of cells and isolating the Islet 1-expressing cells. Alternatively, or in addition, the methods can further involve contacting the population of cells with an agent that promotes Islet1 expression. In addition, the methods can further include, for example, contacting cells that co-express Islet1 and HCN4 with an agent that down regulates Islet1 expression.

In another aspect, the invention provides methods of isolating cardiovascular stem cells. The methods include, for example, isolating cells that express Islet1 but not HCN4 from a population of cells, such as a population of stem and/or progenitor cells. The methods can involve, for example, detecting HCN4 expression in the population of cells and isolating the cells that do not express HCN4. The methods can further involve, for example, detecting Islet1 expression in the population of cells and isolating the Islet1-expressing cells. Alternatively, or in addition, the methods can further involve contacting the population of cells with an agent that promotes Islet 1 expression.

In another aspect, the invention provides methods of differentiating HCN4-expressing progenitor cells. The HCN4-expressing progenitor cells can further express at least one stem cell marker, such as Islet1, Nkx2.5, or Tbx18. The methods can comprise, for example, contacting the HCN4-expressing progenitor cells with an agent that down regulates expression of a stem cell marker, such as Islet1, Nkx2.5, or Tbx18. Alternatively, or in addition, the methods can comprise plating HCN4-expressing progenitor cells in the absence of a feeder cell layer and/or stimulating ErbB2 and/or ErbB4 signaling. The methods can further comprise assessing a phenotype of the HCN4-expressing progenitor cells, for example, after stimulating their differentiation. The methods can be performed in vitro or in vivo.

In another aspect, the invention provides differentiated cells derived from CCS progenitor cells or cardiovascular stem cells. The CCS progenitor cells can be HCN4+ CCS progenitor cells, as described herein. The cardiovascular stem cells can be Islet1+, HCN4-cardiovascular stem cells, as described herein. The differentiated cells can be cardiomyocytes and/or CCS cells, such as SA node cells, AV node cells, His-Purkinje fiber cells, or mixtures thereof.

In another aspect, the invention provides methods of evaluating the effect of a test compound upon HCN4-expressing progenitor cell differentiation. The methods can comprise, for example, contacting HCN4-expressing cells with the test compound under conditions where the HCN4-expressing progenitor cells exhibit little differentiation and assessing whether the test compound stimulates more differentiation. Alternatively, the methods can comprise, for example, contacting HCN4-expressing progenitor cells with the test compound under conditions where the HCN4-expressing progenitor cells would exhibit significant differentiation and assessing whether the test compound stimulates more or less differentiation. The assessment of differentiation can be visual, such as looking at the HCN4-expressing progenitor cells under a microscope to determine whether they have differentiated into cells that resemble SA node cells, AV node cells, or His-Purkinje fiber cells. Alternatively, or in addition, the assessment of differentiation can involve obtaining measurements of membrane depolarization and/or cell contraction.

In another aspect, the invention provides methods of evaluating the toxicity of a test compound. The evaluation can be of the test compound's affect upon the behavior of HCN4-expressing cells. The HCN4-expressing cells be derived from HCN4-expressing progenitor cells, as described herein. The behavior analyzed can be membrane depolarization and/or cell contraction. The evaluation can be visual, such as looking at the HCN4-expressing cells under a microscope to determine whether the test compound changes a physical property of the cells, such as coordinated contraction. Alternatively, or in addition, the evaluation can be a measurement, such as a measurement of membrane depolarization and/or cell contraction. The test compound can be a potential therapeutic agent, such as a small molecule drug, protein, nucleic acid, lipid, hormone, steroid, carbohydrate, etc.

In another aspect, the invention provides methods of treating heart disease in a patient. The methods can comprise delivering cardiovascular stem cells to a region in the patient where the stem cells can function to ameliorate conditions associated with the heart disease, such as in or around the coronary arteries. The heart disease can be coronary artery disease, chronic myocardial ischemia, congestive heart failure, cardiomyopathy, or heart attack. The cardiovascular stem cells can express Islet1 but not HCN4, as described herein. The patient can be a mammal, such as a human.

In another aspect, the invention provides methods of treating heart arrhythmia in a patient. The methods can comprise ablating HCN4-expressing cells in the heart of the patient. The ablation can be achieved by radiofrequency ablation. The methods can further comprise the identification of HCN4-expressing cells, wherein the identification helps to locate the cells that will be ablated. The identification of HCN4-expressing cells can be accomplished using an agent that specifically binds to HCN4 protein. The HCN4-expressing cells can be located outside of the CCS. The patient can be a mammal, such as a human.

In yet another aspect, the invention provides methods for generating an HCN4 lineage-traced cell. The methods can include contacting an undifferentiated progenitor cell that expresses HCN4 with an agent that activates or enhances expression of HCN4 in the cell. Activation or enhancement of HCN4 expression can cause the cell to differentiate into a cell selected from the group consisting of cardiomyocyte or cardiac conduction system (CCS) cell. The undifferentiated progenitor cell can be an epicardial cell, a primary heart field cell, or a secondary heart field cell.

In yet another aspect, the invention provides methods for generating an HCN4 and Islet1 lineage-traced cell. The methods can include contacting an undifferentiated progenitor cell that expresses HCN4 and Islet1 with an agent that activates or enhances expression of HCN4 and Islet1 in the cell. Activation or enhancement of HCN4 and Islet1 expression can cause the cell to differentiate into a cell selected from the group consisting of cardiomyocyte or CCS cell, such as a sinoatiral node cell. The undifferentiated progenitor cell may be an epicardial cell, a primary heart field cell, or a secondary heart field cell.

In yet another aspect, the invention provides methods for treating ischemic disease in a subject. The ischemic disease can be associated with arrhythmia. The methods can include administering to a subject in need thereof an agent that activates or enhances expression of HCN4. The agent can further activate or enhance the expression of Islet1 and/or Tbx 18.

### BRIEF DESCRIPTION OF THE DRAWINGS

FIGS. 1A and 1B are pictorial diagrams showing generation of a HCN4-H2B-EGFP Knock-in Mouse. (a) Targeting strategy. Genomic locus of HCN4 is shown on top, the targeting construct shown in the center, and the locus after recombination shown at the bottom. A knock-in cassette composed of H2B-EGFP followed by FRT-mclNeo was introduced at a Sall site located before the endogenous ATG. The same strategy was used to generate HCN4-CreET2 and nLacZ mice. (b) DNAs from neomycin-resistant ES clones were digested with EcoRV and analyzed by Southern Blot. Wild-type (WT) is 14.8 kb and targeted alleles, 6.9 kb.

FIGS. 2A to 2G are pictorial diagrams showing expression of HCN4-H2B-EGFP in the heart during early heart development. At E8.5, HCN4-EGFP is expressed in the regions of the inflow tract, the sinus venosus (see FIGS. 2A-C, 2F, and 2G) and in the heart tube (see FIGS. 2A-2E). Few if any HCN4-EGFP cells are found in the outflow tract (OFT) region (see FIGS. 2A, 2B and 2D, 2E). DAPI counterstaining is shown in FIGS. 2E and 2G. Arrows indicate positive staining. LV: left ventricle; RV: right ventricle; SV, sinus venosus; OFT, outflow tract

FIGS. 3A to 3F are pictorial diagrams showing expression of HCN4-H2B-EGFP in adult heart. In adult (4-month old), expression of HCN4-EGFP marks all the central conduction system components, including the SAN (see FIGS. 3A, 3B, 3C), CT (see FIG. 3C) and AVN (see FIGS. 3D, 3E). HCN4-EGFP is also expressed in the His-Purkinje fiber and tricuspid, the HB (see FIG. 3E), in the right half wall of the AS that interconnects the SAN and AVN (see FIG. 3D), and in the CS (see FIG. 3F). Arrows indicate positive staining. SAN, sinoatrial node; AVN, atrioventricular node; HB, His Bundle; AS, atrial septum; CS, coronary septum.

FIGS. 4A to 4F are pictorial diagrams showing expression of HCN4-nLacZ. At E8.5, HCN4-nLacZ is expressed in the region of the IFT, AV canal and the ventricle, but not in the OFT (see FIGS. 4A and 4B). At postnatal stage (P2-4), HCN4-nLacZ marks all the components of cardiac condution system, including SAN, AVN, His-Purkinje fibers (see FIG. 4C), similar to the expression pattern of CCS-LacZ (see FIG. 4E). Arrows and arrowheads indicated positive staining. IFT, in flow tract.

FIGS. 5A to 5C are pictorial diagrams showing lineage analysis of HCN4 expression in the developing cardiac conduction system. FIGS. 5A and 5B show E15.5 embryos stained with X-gal, in whole mount and sections. FIG. 5C shows X-gal staining of the P7 heart. LA, left atria; RA, right atria; LV, left ventricle; RV, right ventricle. Arrows indicated positive staining.

FIGS. 6A to 6D are pictorial diagrams showing coimmunostaining of Islet1 and HCN4 in SAN and AVN. At 11.5, strong coimmunostaining of Islet1 and HCN4 was observed in the region of SAN and a few cells in the region of the AVN. (FIG. 6A) HCN4-EGFP; (FIG. 6B) Islet1-Red; (FIG. 6C) merger of A and B; and (FIG. 6D) DAPI staining. The regions of the SAN, AVN and atria septum (AS) are indicated by arrows.

FIGS. 7A to 7H are pictorial diagrams showing coexpression of HCN4-H2B-EGFP with ErbB2 and ErbB4. At E8.5, HCN4-EGFP is expressed in cardiomyocytes mainly in common atria and presumptive left ventricle (see FIGS. 7A and 7E). ErbB2 (FIG. 7B) and ErbB4 (FIG. 7F) are extensively expressed in cardiomyocytes both in atria and ventricles that are coexpressed with HCN4 (FIGS. 7C and 7G). FIGS. 7D and 7H show DAPI countstaining. Arrows indicated co-localization. A, atria; LV, left ventricle; RV, right ventricle.

FIGS. 8A and 8B are pictorial diagrams showing an in utero ultrasound biomicroscopy (UBM) Doppler of control (TnT +/+;Isl loxP/+) (FIG. 8A) and knockout (TnT cre/+;Isl loxP/loxP) embryos (FIG. 8B) at stage E10.5. As shown in FIG. 8A, diastolic E and A waves can be visualized at a typical heart rate of 154 bpm at this embryonic stage. FIG. 8B shows that the mutant embryo heart rate is dramatically slower (14 bpm).

FIGS. 9A to 9D are graphical diagrams showing data from electrophysiological characterization of normal adult and neonatal mice. FIG. 9A shows typical surface ECGs (lead II) of adult (upper panel) and neonatal (lower panel) mice. FIG. 9B shows that, when an adult mouse heart is dissected and maintained using a Langendorff perfusion system at 37° C., normal sinus rhythm and AV conduction is preserved. Epicardial recordings using bipolar electrodes with electrode spacing 1-2mm are shown in sinus rhythm (upper panel) and with pacing from the left atrium (lower panel). Right atrial channel is shown in blue and left ventricular channel is shown in red. Sinus cycle length (SCL) can be recorded (172±21 ms) as well as AV delay (42±7 ms) and sinus nodal recovery time (SNRT) after cessation of pacing (S1) and return of sinus nodal activity. FIG. 9C shows that, with progressively shorter basic pacing cycle length, Wencke-Bach (WB) AV block is observed in normal hearts (upper panel) and WB cycle length can be recorded (98±13 ms). Decremental AV conduction is also observed when progressively shorter extrastimulus (S2) is given with a basic drive train (S1) (lower panel). FIG. 9D shown this relationship, A2-V2 vs. S1-S2, for normal mouse hearts.

FIGS. 10A to 10D are graphical diagrams showing quantification of FACS Data. Left panel show: Y-axis is the dead channel intensity and X-axis is green channel intensive. Right panel: Y-axis max number of cells with relation to green GFP intensity.

FIG. 11 is a pictorial diagram providing confirmation that runxl is a downstream target of Islet1 in sensory ganglia. Sections of trigeminal (TG) and dorsal root ganglia (DRG) at E11.5 in wildtype (left panels) and wnt1-cre;isl1 mutants (right panels) were stained with antibody to Runx 1. Results demonstrate decreased Runx 1 in mutant sensory ganglia, consistent with microarray data. NT=neural tube.

FIG. 12 is an exemplary human HCN4 gene sequence (SEQ ID NO: 1) as provided by GenBank accession no. NM_{—}005477.

FIG. 13 is an exemplary human Islet1 gene sequence (SEQ ID NO: 2) as provided by GenBank accession no. NM_{—}002202.2.

FIG. 14 is an exemplary human Nkx2.5 gene sequence (SEQ ID NO: 3) as provided by GenBank accession no. NM_{—}004387.2.

FIG. 15 an exemplary human Tbx18 gene sequence (SEQ ID NO: 4) as provided by GenBank accession no. NM_{—}001080508.

FIG. 16 an exemplary human flk1 gene sequence (SEQ ID NO: 5) as provided by GenBank accession no. NM_{—}010612.

### DETAILED DESCRIPTION OF THE INVENTION

The present invention provides isolated stem cells and progenitor cells, including HCN4-expressing CCS progenitor cells and Islet1-expressing cardiovascular stem cells that do not express HCN4. The invention further provides differentiated cell types derived from the CCS progenitor cells and cardiovascular stem cells of the invention. In addition, the invention provides methods of differentiating the cells of the invention, methods for screening for agents that affect the differentiation of the cells of the invention, methods for evaluating the potential toxicity of drugs using the cells of the invention, cell therapy using the cells of the invention, and methods of treating heart arrhythmia by ablating HCN4-expressing cells.

Accordingly, in one aspect, the invention provides isolated stem cells and progenitor cells capable of giving rise to (i.e., differentiating into) specialized cell types found within a heart. As used herein, a “stem cell” is a cell that is (1) capable of self-renewal, (2) unspecialized, and (3) capable of giving rise to multiple specialized cell types. As used herein, a “progenitor cell” is a cell that, in terms of differentiation, is between a stem cell and a specialized cell. Thus, a progenitor cell is typically capable of limited self-renewal; is unspecialized, but closer to being specialized than a stem cell; and gives rise to fewer specialized cell types (e.g., one or a small number of related cell types). Although stem cells typically give rise to progenitor cells and progenitor cells typically give rise to specialized cell types, progenitor cells can sometimes revert to a stem cell state (e.g., when the appropriate environmental cues are present).

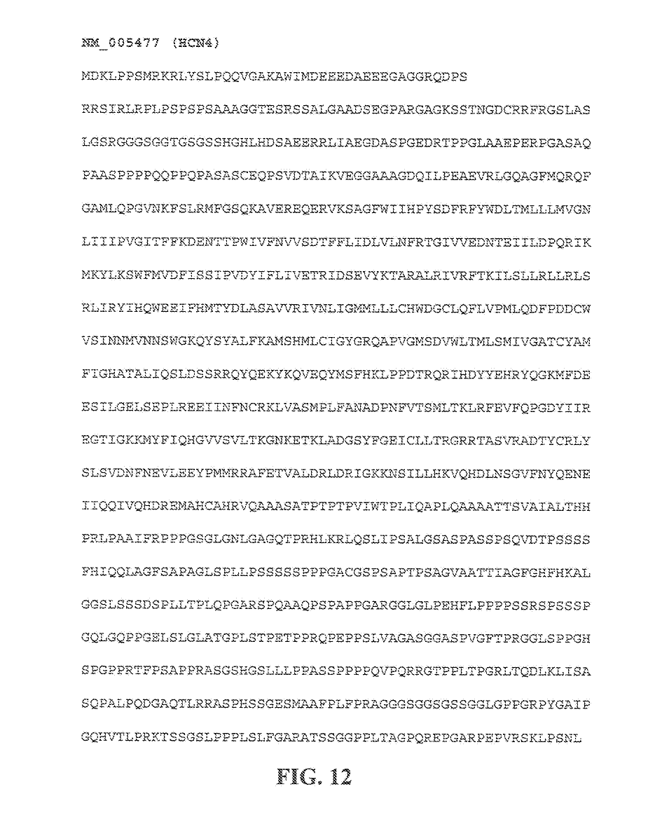

In certain embodiments, the isolated progenitor cells are CCS progenitor cells that express HCN4. As used herein, “HCN4” refers to a nucleic acid or peptide sequence corresponding to human Hyperpolarization-activated, Cyclic Nucleotide gated (HCN) channel 4, or an ortholog thereof. An exemplary human HCN4 gene sequence is provided by GenBank sequence NM_{—}005477 (SEQ ID NO: 1; FIG. 12). Non-human orthologs of HCN4, including the mouse, rat, and chicken orthologs, are identified in NCBI HomoloGene:3997. In certain embodiments, at least 75%, 80%, 85%, 90%, 95%, 98%, or more of the cells in the isolated population of CCS progenitor cells express HCN4 (i.e., are HCN4+).

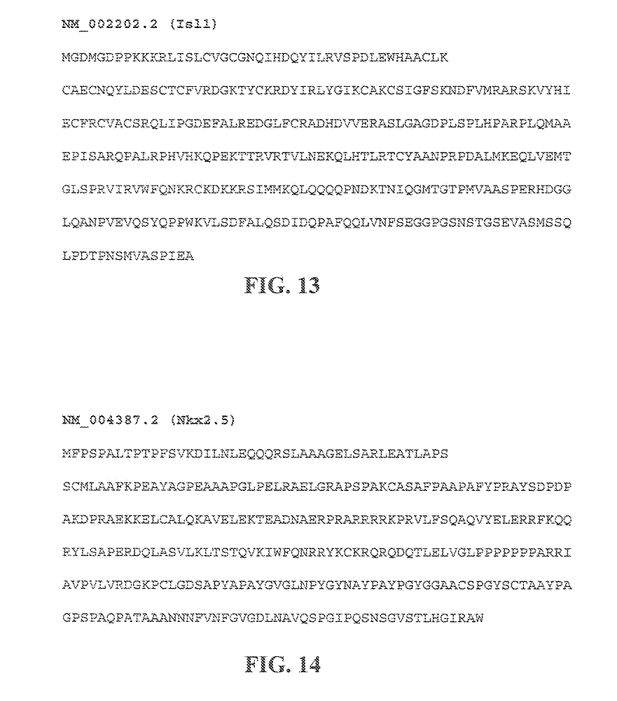

In certain embodiments, the isolated progenitor cells are CCS progenitor cells that co-express HCN4 and Islet1. As used herein, “Islet1” refers to a nucleic acid or peptide sequence corresponding to human Islet1, or an ortholog thereof. An exemplary human Islet1 gene is provided by GenBank sequence NM_{—}002202.2 (SEQ ID NO: 2; FIG. 13). Non-human orthologs of Islet1, including the chimpanzee, canine, bovine, mouse, rat, and chicken homologs, are identified in NCBI HomologGene: 1661. In certain embodiments, at least 75%, 80%, 85%, 90%, 95%, 98%, or more of the cells in the isolated population of CCS progenitor cells co-express HCN4 and Islet1 (i.e., are HCN4+/Islet1+). In certain embodiments, the CCS progenitor cells that co-express HCN4 and Islet1 do not express Nkx2.5.

In certain embodiments, the isolated progenitor cells are CCS progenitor cells that co-express HCN4 and Nkx2.5. As used herein, “Nkx2.5” refers to a nucleic acid or peptide sequence corresponding to human Nkx2.5, or an ortholog thereof. An exemplary human Nkx2.5 gene is provided by GenBank sequence NM_{—}004387.2 (SEQ ID NO: 3; FIG. 14). Non-human orthologs of Nkx2.5, including the chimpanzee, canine, bovine, mouse, rat, and chicken homologs, are identified in NCBI HomologGene: 3230. In certain embodiments, at least 75%, 80%, 85%, 90%, 95%, 98%, or more of the cells in the isolated population of CCS progenitor cells co-express HCN4 and Nkx2.5 (i.e., are HCN4+/Nkx2.5+). In certain embodiments, the CCS progenitor cells that co-express HCN4 and Nkx2.5 do not express Islet1.

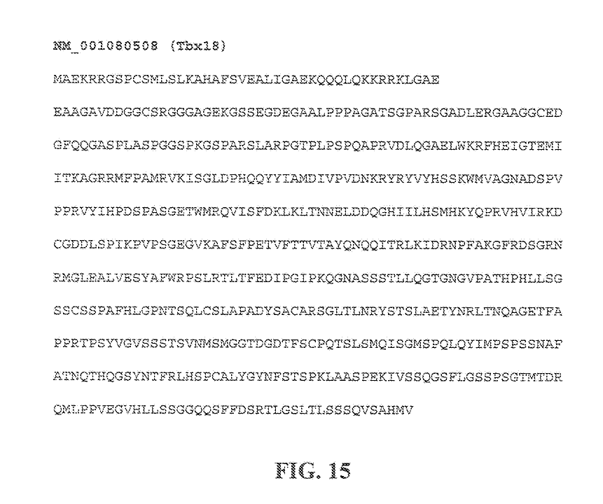

In certain embodiments, the isolated progenitor cells are CCS progenitor cells that co-express HCN4 and Tbx18. As used herein, “Tbx18” refers to a nucleic acid or peptide sequence corresponding to human Tbx18, or an ortholog thereof. An exemplary human Tbx18 gene is provided by GenBank sequence NM_{—}001080508 (SEQ ID NO: 4; FIG. 15). Non-human orthologs of Tbx18, including the chimpanzee, canine, bovine, mouse, rat, and chicken homologs, are identified in NCBI HomologGene: 11384. In certain embodiments, at least 75%, 80%, 85%, 90%, 95%, 98%, or more of the cells in the isolated population of CCS progenitor cells co-express HCN4 and Tbx18 (i.e., are HCN4+/Tbx18+). In certain embodiments, the CCS progenitor cells that co-express HCN4 and Tbx18 do not express Islet1.

As used herein, the term “express” refers to the presence of a particular gene transcript (i.e., RNA transcript) and/or corresponding protein within a cell. In certain embodiments, a cell expresses a particular gene (e.g., HCN4, Islet1, Nkx2.5, Tbx18, flk1, etc.) when there is at least one copy of a corresponding RNA transcript or protein product in the cell. In other embodiments, a cell expresses a particular gene when the corresponding RNA transcript or protein product levels exceeds background levels (e.g., background levels associated with a particular assay system that can be used to evaluate expression levels, such as fluorescence activated cell sorting (FACS)).

In certain embodiments, the isolated CCS progenitor cells can differentiate to form SA node cells, AV node cells, or His-Purkinje fiber cells. In other embodiments, the CCS progenitor cells can differentiate to form cardiomyocytes. In certain embodiments, the isolated CCS progenitor cells co-express HCN4 and Islet1 and can differentiate to form SA node cells. In certain embodiments, the isolated CCS progenitor cells co-express HCN4 and Nkx2.5 and can differentiate to form AV node cells.

In certain embodiments, the isolated stem and/or progenitor cells are cardiovascular stem and/or progenitor cells that express HCN4 and a stem cell maker selected from the group consisting of Islet1, Nkx2.5, and Tbx18. In certain embodiments, at least 75%, 80%, 85%, 90%, 95%, 98%, or more of the cells in the isolated population of cardiovascular stem and/or progenitor cells co-express HCN4 and a stem cell maker selected from the group consisting of Islet1, Nkx2.5, and Tbx18. In certain embodiments, at least 75%, 80%, 85%, 90%, 95%, 98%, or more of the cells in the isolated population of cardiovascular stem and/or progenitor cells co-express HCN4 and Islet1. In certain embodiments, at least 75%, 80%, 85%, 90%, 95%, 98%, or more of the cells in the isolated population of cardiovascular stem and/or progenitor cells co-express HCN4 and Nkx2.5. In certain embodiments, at least 75%, 80%, 85%, 90%, 95%, 98%, or more of the cells in the isolated population of cardiovascular stem and/or progenitor cells co-express HCN4 and Tbx18. In certain embodiments, the HCN4-expressing cardiovascular stem and/or progenitor cells can differentiate to form cardiomyocytes. In certain embodiments, the HCN4-expressing cardiovascular stem and/or progenitor cells can differentiate to form CCS cells, such as SA node cells, AV node cells, or His-Purkinje fiber cells.

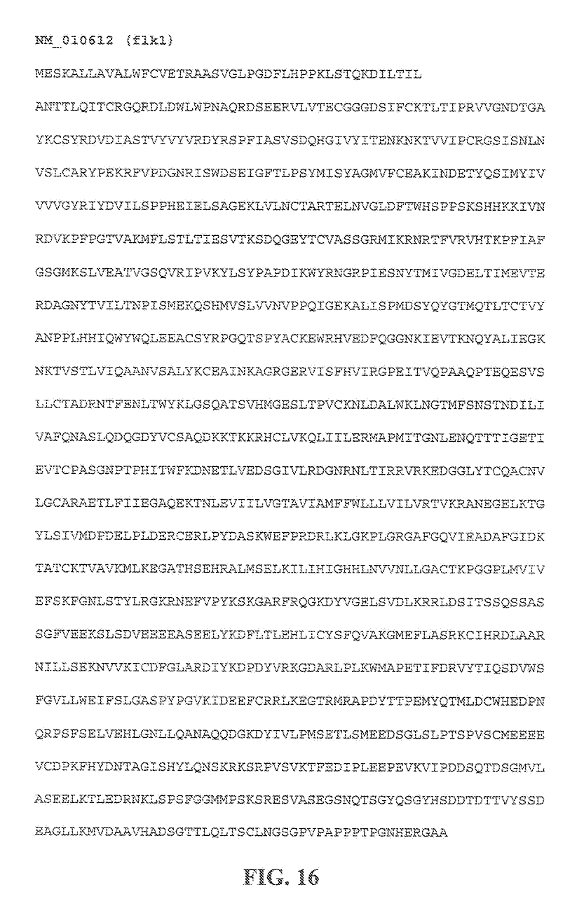

In certain embodiments, the isolated stem and/or progenitor cells are cardiovascular stem cells that express Islet1 but not HCN4. In certain embodiments, at least 75%, 80%, 85%, 90%, 95%, 98%, or more of the cells in the isolated population of cardiovascular stem cells express Islet1 but not HCN4. In certain embodiments, the cardiovascular stem cells that express Islet1 but not HCN4 also express Nkx2.5, flk1, and/or LeX/SSEA1/CD15. As used herein, “flk1” refers to a nucleic acid or peptide sequence corresponding to human flk1 (also known as KDR, for “kinase insert domain receptor”), or an ortholog thereof An exemplary human flk1 gene sequence is provided by GenBank sequence NM_{—}010612 (SEQ ID NO: 5; FIG. 16). Non-human orthologs of flk1, including the chimpanzee, canine, bovine, mouse, and chicken homologs, are identified in NCBI HomoloGene:55639. In certain embodiments, at least 75%, 80%, 85%, 90%, 95%, 98%, or more of the cells in the isolated population of cardiovascular stem cells co-express Islet1 and Nkx2.5, flk1, or LeX/SSEA1/CD15, but do not express HCN4. In certain embodiments, the cardiovascular stem cells that express Islet1 but not HCN4 can differentiate to form cardiomyocytes and/or vascular cells (e.g., endocardium or smooth muscle cells).

In another aspect, the invention provides methods of isolating CCS progenitor cells. The CCS progenitor cells can be any type of CCS progenitor cell described herein. In certain embodiments, the CCS progenitor cells express HCN4. In other embodiments, the CCS progenitor cells co-express HCN4 and a stem cell marker. As used herein, the term “stem cell marker” refers to a nucleic acid or protein sequence associated with stem cell identity. In certain embodiments, the stem cell marker is a transcription factor (e.g., a transcription factor active in one or more types of stem cells and that promotes a stem cell phenotype, such as self-renewal and/or a nonspecialized state). In certain embodiments, the stem cell marker is selected from the group consisting of Islet1, Nkx2.5, and Tbx18. In certain embodiments, the stem cell marker is a cell-surface receptor, such as a receptor that contains a kinase domain (e.g., flk1) or directly interacts with a kinase. In certain embodiments, the stem cell marker binds to or is associated with the cell surface of a stem cell (e.g., LeX/SSEA1/CD15).

In certain embodiments, the CCS progenitor cells of the invention (e.g., HCN4-expressing progenitor cells) are capable of differentiating into CCS cell types, such as SA node cells, AV node cells, His-Purkinje fiber cells, or a combination thereof. In other embodiments, the CCS progenitor cells are capable of differentiating into cardiomyocytes.

CCS progenitor cells of the invention can be isolated from a population of cells. The population of cells can be a population that consists of or comprises stem and/or progenitor cells. For example, in certain embodiments, the population of cells is or comprises a population of embryonic stem cells. In other embodiments, the population of cells is or comprises a population of adult stem cells. In certain embodiments, the population of cells is or comprises a population of mesenchymal stem cells (e.g., bone marrow derived stem cells, adipose-derived stem cells, dermal stem cells, etc.). In certain embodiments, the population of cells is from an embryonic, post-natal, adolescent, or adult animal, such as a mammal (e.g., a human, primate, monkey, mouse, rat, dog, cat, horse, cow, pig, etc.) or a bird (e.g., a chicken). In certain embodiments, the population of cells is from a mesodermal tissue, heart tissue, epicardial tissue, serosal mesothelial tissue, adipose tissue, dermal tissue, bone marrow, or umbilical cord.

In certain embodiments, the methods of isolating CCS progenitor cells comprise sorting the population of cells (e.g., the population of stem and/or progenitor cells) based upon HCN4 expression levels. In certain embodiments, HCN4 expression level is determined by detecting HCN4 protein. For example, in certain embodiments, HCN4 protein is detected using an antibody to HCN4, such as an antibody that binds to an extracellular portion of HCN4 (e.g., an extracellular peptide of HCN4 or an extracellular site of modification on HCN4, such as a glycosylation site). In certain embodiments, the antibody is fluorescently labeled. In other embodiments, HCN4 protein is detected by means of a HCN4-specific binding agent. The binding agent can be a protein (e.g., a single-chain antibody), peptide, small molecule, or fluorescently labeled derivative thereof.

In certain embodiments, HCN4 expression level is determined by detecting transcription from an HCN4 promoter. The HCN4 promoter can be located at the HCN4 gene locus, inserted into another region of the genome, or on a plasmid. In certain embodiments, for example, a reporter gene is integrated into the HCN4 gene locus such that it is transcribed whenever the HCN4 promoter is active (e.g., a knock-in reporter). In other embodiments, the reporter gene is operably linked to an HCN4 promoter to form a construct and the construct is integrated into another location in the genome or into a plasmid. In certain embodiments, the reporter gene codes for a fluorescent protein, such as GFP, EGFP, or H2B-EGFP. In other embodiments, the reporter gene codes for a recombinase, such as Cre recombinase or tomoxifen-inducible Cre recombinase. In still other embodiments, the reporter gene codes for an enzyme, such as β-galactosidase (e.g., a lacZ reporter gene).

In certain embodiments, the methods of isolating CCS progenitor cells comprise sorting the population of cells (e.g., the population of stem and/or progenitor cells) based upon HCN4 expression levels and the expression levels of at least one stem cell marker. In certain embodiments, the at least one stem cell marker is selected from the group consisting of Islet1, Nkx2.5, Tbx18, flk1, and LeX/SSEA1/CD15.

In certain embodiments, the expression level of a stem cell marker is determined by detecting the stem cell marker protein (e.g., Islet1, Nkx2.5, Tbx18, or flk1 protein) or carbohydrate (e.g., LeX/SSEA1/CD15). For example, in certain embodiments, the stem cell marker protein is detected using an antibody to the protein or carbohydrate. In certain embodiments, the antibody binds to an extracellular portion of the stem cell marker protein (e.g., an extracellular peptide from the protein or an extracellular site of modification on the protein, such as a glycosylation site). In other embodiments, the antibody binds to an intracellular portion of the stem cell maker protein (e.g., in permeabilized cells). In certain embodiments, the antibody is fluorescently labeled. In other embodiments, the stem cell marker protein is detected by means of a specific binding agent. The binding agent can be a protein (e.g., a single-chain antibody), peptide, small molecule, or fluorescently labeled derivative thereof.

In certain embodiments, the expression level of a stem cell marker is determined by detecting transcription from an stem cell marker promoter (e.g., an Islet1, Nkx2.5, Tbx18, or flk1 promoter). The stem cell marker promoter can be the endogenous promoter located at the stem cell marker gene locus. Alternatively, the stem cell marker promoter can be inserted into another region of the genome, or on a plasmid. In certain embodiments, for example, a reporter gene is integrated into the stem cell marker gene locus such that it is transcribed whenever the stem cell marker promoter is active (e.g., a knock-in reporter). In other embodiments, the reporter gene is operably linked to a stem cell marker promoter to form a construct and the construct is integrated into another location in the genome or into a plasmid. In certain embodiments, the reporter gene codes for a fluorescent protein, such as GFP, EGFP, or H2B-EGFP. In other embodiments, the reporter gene codes for a recombinase, such as Cre recombinase or tomoxifen-inducible Cre recombinase. In still other embodiments, the reporter gene codes for an enzyme, such as β-galactosidase (e.g., a lacZ reporter).

In certain embodiments, the expression level of a stem cell marker is determined by detecting transcription from a promoter activated by the stem cell marker. For example, in certain embodiments, the stem cell marker is a transcription factor which, when active, promotes transcription from target promoters. The target promoter can be operably linked to a reporter gene that codes for a fluorescent protein, recombinase, or enzyme, as described above.

In certain embodiments, the expression level of a stem cell marker is determined by detecting an endogenous gene product, such as a cell surface protein whose expression is dependent upon the activity of the stem cell marker. Thus, in certain embodiments, stem cell marker activity (e.g., transcriptional activity) induces expression of an endogenous gene and/or protein, wherein the expression level of the endogenous gene and/or protein can be tested by any of the methods disclosed herein, including the use of antibodies e.g., fluorescently-labeled antibodies), or knock-in reporters.

In certain embodiments, the methods of isolating CCS progenitor cells comprise sorting the population of cells (e.g., the population of stem and/or progenitor cells) based upon HCN4 expression levels and then detecting expression of a stem cell marker in a subset of the sorted cells. For example, in certain embodiments, a subset of the isolated CCS progenitor cells are tested for expression of at least one stem cell marker in order to confirm the presence of CCS progenitor cells and/or determine the percentage of CCS progenitor cells in the isolate. In certain embodiments, the at least one stem cell marker is selected from the group consisting of Islet1, Nkx2.5, Tbx18, flk1, and LeX/SSEA1/CD15.

The expression level of a gene or protein, such as HCN4, a stem cell marker (e.g., Islet1, Nkx2.5, Tbx18, flk1, LeX/SSEA1/CD15), a reporter, or an endogenous gene whose expression is dependent upon the activity of a stem cell marker, can be determined using fluorescence activated cell sorting (FACS). In certain embodiments, gene or protein expression levels are determined for a population of cells and the cells are sorted by FACS based upon the results of the evaluation or measurement. In certain embodiments, the FACS sorting separates cells that have a moderate or high expression level of a particular gene or protein from cells that have low or negligible expression levels of the particular gene or protein. In other embodiments, the FACS sorting separates cells that have a high expression level of a particular gene or protein from cells that have moderate to negligible expression levels of the particular gene or protein. Persons skilled in the art will understand that appropriate cut offs for sorting cells into classes of moderate to high expressers vs. low to negligible expressers or high expressers vs. moderate to negligible expressers will depend upon the particular gene or protein being evaluated, the cells being sorted, and the amount of background in the system. However, persons skilled in the art will also understand that routine experimentation can be employed to identify such cutoffs.

FACS sorting of cells can be accomplished using antibodies to specific proteins or antigens of interest, wherein the antibodies are fluorescently labeled. Alternatively, the antibodies can be detected by means of a fluorescently-labeled secondary antibody. Similarly, expression of reporter genes can be used to FACS sort cells. Expression of reporters that encode fluorescent proteins allows for use of FACS in the identification and sorting of cells that express the reporter. Similarly, cells that express reporters encoding enzymes, such as β-galactosidase, can be detected by providing a substrate for the enzyme that fluoresces upon enzymatic processing (e.g., 6-O-galactopyranosyl-luciferin, which can be cleaved by β-galactosidase to yield a fluorescent product). Cells that express reporter genes encoding recombinases can further include a second reporter gene that can be measured in a fluorescence-based assay. For example, the secondary reporter can encode an enzymatic molecule such as β-galactosidase, allowing for fluorescence-based detection, as described above (e.g., in the case of Cre recombinase, a Rosa26 promoter linked to lacZ can be used as the secondary reporter). In each instance, increased reporter expression translates into increased cellular fluorescence. In certain embodiments, increased cellular fluorescence indicates increased expression of the gene or protein of interest (e.g., HCN4, Islet1, Nkx2.5, Tbx18, or flk1). In certain embodiments, cell size can be used in the FACS analysis to assist in isolating the desired cells. In certain embodiments, the stem and progenitor cells of the invention have, on average, a smaller size as compared to differentiated cell types.

In certain embodiments, the methods of isolating CCS progenitor cells further comprise growing the population of cells (e.g., the population of stem and/or progenitor cells) on a layer of feeder cells or with conditioned medium or extracellular matrix produced by the feeder cells. The feeder cells can be any type of feeder cells suitable for promoting or maintaining stem cell self-renewal and suppressing differentiation (i.e., promoting or maintaining a nonspecialized stem cell state). In certain embodiments, the feeder cells are cardiac fibroblasts.

In certain embodiments, the methods of isolating CCS progenitor cells further comprise contacting the population of cells with an agent that promotes or maintains stem cell self-renewal and/or suppresses differentiation (i.e., promotes or maintains a nonspecialized stem cell state). In certain embodiments, the agent is or comprises a Wnt protein (e.g., Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16) or a Wnt signaling pathway agonist (e.g., a protein, peptide, or small molecule activator of a Wnt signaling pathway, such as 2-amino-4-[3,4(methylenedioxy) benzyl-amino]-6-(3-methoxyphenyl)pyrimidine). In certain embodiments, the agent is or comprises a GSK-3β antagonist (e.g., a protein, peptide, or small molecule inhibitor of GSK-3β, such as 6-bromoindirubin-3′oxime (BIO)). In certain embodiments, the agent is or comprises an activin protein or TGF-β/activin signaling pathway agonist (e.g., a protein, peptide, or small molecule activator of a TGF-β/activin signaling pathway). In certain embodiments, the agent is or comprises a BMP signaling pathway antagonist (e.g., a protein, such as Chordin, Noggin, or Dysmorphin, a peptide, or a small molecule inhibitor of the BMP signaling pathway). In certain embodiments, the agent is or comprises a Smad 1, Smad 5, or Smad 8 antagonist (e.g., a protein, peptide, or small molecule inhibitor of Smad 1, Smad 5, or Smad 8 function).

In another aspect, the invention provides methods of isolating SA node cells or progenitors thereof. In certain embodiments, the methods comprise contacting a population of cells with an agent that promotes or maintains Islet1 expression, and isolating cells that express HCN4 from the population. In certain embodiments, the SA node cells or progenitors thereof co-express HCN4 and Islet1.

The population of cells can be a population that consists of or comprises stem and/or progenitor cells. For example, in certain embodiments, the population of cells is or comprises a population of embryonic stem cells. In other embodiments, the population of cells is or comprises a population of adult stem cells. In certain embodiments, the population of cells is or comprises a population of mesenchymal stem cells (e.g., bone marrow-derived stem cells, adipose-derived stem cells, dermal stem cells, etc.). In certain embodiments, the population of cells is from an embryonic, post-natal, adolescent, or adult animal, such as a mammal (e.g., a human, primate, monkey, mouse, rat, dog, cat, horse, cow, pig, etc.) or a bird (e.g., a chicken). In certain embodiments, the population of cells is from a mesodermal tissue, heart tissue, epicardial tissue, serosal mesothelial tissue, adipose tissue, dermal tissue, bone marrow, or umbilical cord.

In certain embodiments, the agent that promotes Islet 1 expression is a layer of feeder cells or extracellular matrix and/or conditioned medium produced by the feeder cells. The feeder cells can be any type of feeder cells suitable for promoting or maintaining stem cell self-renewal and suppressing differentiation (i.e., promoting or maintaining the nonspecialized stem cell state). In certain embodiments, the feeder cells are cardiac fibroblasts.

In certain embodiments, the agent that promotes or maintains Islet1 expression is an FGF protein or an FGF signaling pathway agonist. In certain embodiments, the agent that promotes or maintains Islet1 expression is a Wnt protein (e.g., Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, Wnt16) or a Wnt signaling pathway agonist (e.g., a protein, peptide, or small molecule activator of a Wnt signaling pathway, such as 2-amino-4-[3,4(methylenedioxy) benzyl-amino]-6-(3-methoxyphenyl)pyrimidine)). In certain embodiments, the agent that promotes or maintains Islet1 expression is or comprises a GSK-3β antagonist (e.g., a protein, peptide, or small molecule inhibitor of GSK-3β, such as 6-bromoindirubin-3′oxime (BIO)). In certain embodiments, the agent that promotes or maintains Islet1 expression is or comprises an activity protein or a TGF-β/activin signaling pathway agonist (e.g., a protein, peptide, or small molecule activator of a TGF-β/activin signaling pathway). In certain embodiments, the agent that promotes or maintains Islet1 expression is or comprises a BMP signaling pathway antagonist (e.g., a protein such as Chordin, Noggin, or Dysmorphin, a peptide, or a small molecule inhibitor of BMP signaling). In certain embodiments, the agent that promotes or maintains Islet1 expression is or comprises a Smad 1, Smad 5, or Smad 8 antagonist (e.g., a protein, peptide, or small molecule inhibitor of Smad 1, Smad 5, or Smad 8 function).

In certain embodiments, the methods of isolating SA node cells or progenitors thereof comprise sorting the population of stem and/or progenitor cells based upon HCN4 expression levels. The determination of HCN4 expression levels can be performed by any means described herein.

In certain embodiments, the methods of isolating SA node cells or progenitors thereof comprise sorting the population of stem and/or progenitor cells based upon HCN4 expression levels and Islet1 expression levels. The determination of Islet1 expression levels can be performed by any means described herein.

In certain embodiments, the methods of isolating SA node cells or progenitors thereof can be performed by FACS, as described herein. In certain embodiments, SA node progenitor cells have a higher level of Islet1 expression as compared to SA node cells. In certain embodiments, SA node cells have a higher level of HCN4 expression as compared to SA node progenitor cells.

In certain embodiments, the methods of isolating SA node cells further comprise contacting isolated HCN4-expressing cells with an agent that suppresses or reduces Islet1expression. In certain embodiments, the agent is an FGF signaling pathway antagonist (e.g., a protein, peptide, or small molecule drug, such as SU5402). In certain embodiments, the agent is or comprises a Wnt signaling pathway antagonist (e.g., a protein, peptide, or small molecule that inhibits the activity of a Wnt signaling pathway, such as PKF115-584 (Doughman and Czareth, 2008), or FH535 (Handeli and Simon 2008)). In certain embodiments, the agent is or comprises a TGF-β signaling pathway antagonist (e.g., a protein, such as Cripto, a peptide, or a small molecule (e.g., SB-431542 (Hjelmeland et al., 2004), SD-208 (Zhou and Nguyen, 2008), or IN-1130 (Lee et al., 2008)) that inhibits the activity of a TGF-β signaling pathway). In certain embodiments, the agent is or comprises an activin signaling antagonist (e.g., a protein, peptide, or small molecule that inhibits the activity of an activin signaling pathway). In certain embodiments, the agent is or comprises a Smad2 and/or Smad3 antagonist (e.g., a protein, peptide, or small molecule that inhibits the activity of Smad2 and/or Smad3). In certain embodiments, the agent is or comprises a Smad1, Smad5, and/or Smad8 agonist (e.g., a protein, peptide, or small molecule that stimulates the activity of Smad1, Smad5, and/or Smad8). In certain embodiments, the agent is or comprises a combination of any of the foregoing agents.

In certain embodiments, the methods of isolating SA node cells further comprise contacting isolated HCN4-expressing cells with an agent that stimulates ErbB2 and/or ErbB4 signaling (e.g., an ErbB2 or ErbB4 ligand). In certain embodiments, the methods of isolating SA node cells further comprise plating isolated HCN4-expressing cells in the absence of a feeder cell layer. In certain embodiments, the methods of isolating SA node cells further comprises any combination of (1) contacting isolated HCN4-expressing cells with an agent that suppresses or reduces Islet1 expression, (2) contacting isolated HCN4-expressing cells with an agent that stimulates ErbB2 and/or ErbB4 signaling, and (3) plating isolated HCN4-expressing cells in the absence of a feeder cell layer.

In another aspect, the invention features methods of isolating cardiovascular stem cells. In certain embodiments, the cardiovascular stem cells express Islet1. In certain embodiments, the cardiovascular stem cells express Islet1, but not HCN4. In certain embodiments, the cardiovascular stern cells express Islet1 and Nkx2.5 and/or flk1, but not HCN4.

The cardiovascular stem cells (e.g., stem cells that express Islet1 but not HCN4) are typically isolated from a population of cells. The population of cells can be a population that consists of or comprises stem and/or progenitor cells. For example, in certain embodiments, the population of cells is or comprises a population of embryonic stem cells. In other embodiments, the population of cells is or comprises a population of adult stem cells. In certain embodiments, the population of cells is or comprises a population of mesenchymal stem cells (e.g., bone marrow-derived stem cells, adipose-derived stem cells, dermal stem cells, etc.). In certain embodiments, the population of cells is from an embryonic, post-natal, adolescent, or adult animal, such as a mammal (e.g., a human, primate, monkey, mouse, rat, dog, cat, horse, cow, pig, etc.) or a bird (e.g., a chicken). In certain embodiments, the population of cells is from a mesodermal tissue, heart tissue, epicardial tissue, serosal mesothelial tissue, adipose tissue, dermal tissue, bone marrow, or umbilical cord.

In certain embodiments, the methods of isolating cardiovascular stem cells comprise sorting the population of stem and/or progenitor cells based upon Islet1 expression levels. The determination of Islet1 expression levels can be performed by any means described herein.

In certain embodiments, the methods of isolating cardiovascular stem cells comprise sorting the population of stem and/or progenitor cells based upon HCN4 expression levels. The determination of HCN4 expression levels can be performed by any means described herein.

In certain embodiments, the methods of isolating cardiovascular stem cells comprise sorting the population of stem and/or progenitor cells based upon HCN4 expression levels and Islet1 expression levels.

In certain embodiments, the methods of isolating cardiovascular stem cells can be performed by FACS, as described herein. In certain embodiments, the cardiovascular stem cells are isolated by FACS using a positive selection for Islet1 expression and a negative selection for HCN4 expression. In other embodiments, the methods of isolating cardiovascular stem cells comprise contacting the population of cells from which the cardiovascular stem cells are isolated with an agent that promotes or maintains Islet1 expression, followed by a negative FACS selection for HCN4 expression.

In certain embodiments, the methods of isolating cardiovascular stem cells further comprises contacting the population of cells with an agent that promotes or maintains Islet1 expression. The agent that promotes or maintains Islet1 expression can be any agent described or suggested herein that promotes or maintains Islet1 expression.

In another aspect, the invention provides methods of differentiating CCS progenitor cells (e.g., HCN4-expressing progenitor cells). In certain embodiments, the methods comprise growing the CCS progenitor cells in vitro, in the absence of feeder cells. In other embodiments, the methods comprise contacting CCS progenitor cells with an agent that down regulates the expression of a stem cell marker. In certain embodiments, the stem cell marker is selected from the group consisting of Islet1, Nkx2.5, and Tbx18.

In certain embodiments, the agent that downregulates the expression of a stem cell marker is selected from the group consisting of an FGF signaling pathway antagonist (e.g., SU5402), a Wnt signaling antagonist (e.g., PKF115-584 or FH535), a TGF-β/activin signaling pathway antagonist (e.g., Cripto, SB-431542, SD-208, or IN-1130), an inhibin, a Smad2 antagonist, a Smad3 antagonist, a BMP signaling pathway agonist, a Smad1 agonist, a Smad5 agonist, a Smad8 agonist, or any combination thereof. In other embodiments, the agent that down regulates the expression of a stem cell marker is an agent that stimulates ErbB2 and/or ErbB4 signaling (e.g., an ErbB2 or ErbB4 ligand).

In certain embodiments, the methods of differentiating CCS progenitor cells (e.g., HCN4-expressing CCS progenitor cells) comprise any combination of (1) contacting isolated CCS progenitor cells with an agent that suppresses or reduces Islet1 expression, (2) contacting isolated CCS progenitor cells with an agent that stimulates ErbB2 and/or ErbB4 signaling, and (3) growing CCS progenitor cells in vitro in the absence of feeder cells.

In certain embodiments, the methods of differentiating CCS progenitor cells further comprise evaluating whether the CCS progenitor cells that have, in fact, differentiated. In certain embodiments, the evaluation is visual (e.g., through a microscope). For example, in certain embodiments, the evaluation comprises observing whether the CCS progenitor cells have changed shape and adopted a differentiated cell phenotype (e.g., the phenotype of an SA node cell, AV node cell, or a His-Purkinje fiber cell). In certain embodiments, the differentiated cell phenotype is a cardiomyocyte phenotype (e.g., a cardiomyocyte phenotype in which the cells beat spontaneously). In other embodiments, the evaluation comprises detecting the expression of genes or proteins associated with a differentiated cell phenotype (e.g., the phenotype of an SA node cell, AV node cell, a His-Purkinje fiber cell, or a cardiomyocyte). In other embodiments, the evaluation comprises obtaining measurements of cell differentiation, such as measurements of cell membrane depolarization (e.g., I_{f} or Iₕ current) and/or cellular contraction. Methods for obtaining current measurements from heart cells have been describe, for example, in Stieber et al. (2003), Proc. Natl. Acad Sci USA 100(25):15235-240, the contents of which are incorporated herein in their entirety.

In another aspect, the invention features methods of identifying compounds that stimulate CCS progenitor cell differentiaton. In certain embodiments, the CCS progenitor cells are HCN4-expressing progenitor cells. In certain embodiments, the CCS progenitor cells differentiate into SA node cells, AV node cells, His-Purkinje fiber cells, or any combination thereof. In other embodiments, the CCS progenitor cells differentiate into cardiomyoctyes.

In certain embodiments, the methods comprise contacting CCS progenitor cells (e.g., HCN4-expressing progenitor cells) with a test compound and evaluating whether the test compound induces CCS progenitor cell differentiation. In certain embodiments, the evaluating is visual or experimental, as described herein. In certain embodiments, the test compound is a protein, peptide, carbohydrate, lipid, steroid, or small organic molecule (e.g., a basic, neutral, or acid drug).

In another aspect, the invention features methods of evaluating the toxicity of a test compound. In certain embodiments, the methods comprise contacting CCS cells (e.g., HCN4-expressing cells or cells that co-express HCN4 and Islet1) with a test compound and then assessing whether a phenotype of the CCS cells has changed following the contacting. In certain embodiments, the CCS cells are SA node cells, AV node cells, His-Purkinje fiber cells, or mixtures thereof. In certain embodiments, the contacting occurs in vitro.

In certain embodiments, the assessment is visual (e.g., observing the cells through a microscope to determine whether spontaneous contractions have been disrupted). In other embodiments, the assessment is experimental. For example, in certain embodiments, the evaluation comprises obtaining measurements of CCS cell function, such as measurements of cell membrane depolarization (e.g., I_{f} or Iₕ current) and/or cellular contraction.

In certain embodiments, the test compound is a protein, peptide, carbohydrate, lipid, steroid, or small organic molecule (e.g., a basic, neutral, or acid drug).

In another aspect, the invention features methods for treating a patient suffering from heart disease. In certain embodiments, the methods comprise delivering a population of cardiovascular stem cells to the heart of the patient. The cardiovascular stem cells can be any cardiovascular stem cells described herein. In certain embodiments, the cardiovascular stem cells express Islet1 but not HCN4. In certain embodiments, the heart disease is selected from the group consisting of coronary artery disease, chronic myocardial ischemia, congestive heart failure, cardiomyopathy, and heart attack. In certain embodiments, the patient is an animal, such as a mammal (e.g., a human, primate, monkey, cow, horse, pig, dog, cat, rat, mouse, etc.) or a bird (e.g., a chicken).

In yet another aspect, the invention provides methods of treating a patient having cardiac arrhythmia. In certain embodiments, the methods comprise ablating (e.g., by means of radiofrequency ablation) an HCN4-expressing cell in the heart of the patient. In certain embodiments, the HCN4-expressing cell is located outside the CCS of the patient. In certain embodiments, the HCN4-expressing cells is identified by means of an HCN4-binding agent. In certain embodiments, the patient is an animal, such as a mammal (e.g., a human, primate, monkey, cow, horse, pig, dog, cat, rat, mouse, etc.) or a bird (e.g., a chicken).

The following examples are intended to illustrate but not limit the invention.

### Example 1

### HCN4 Expression in the Heart

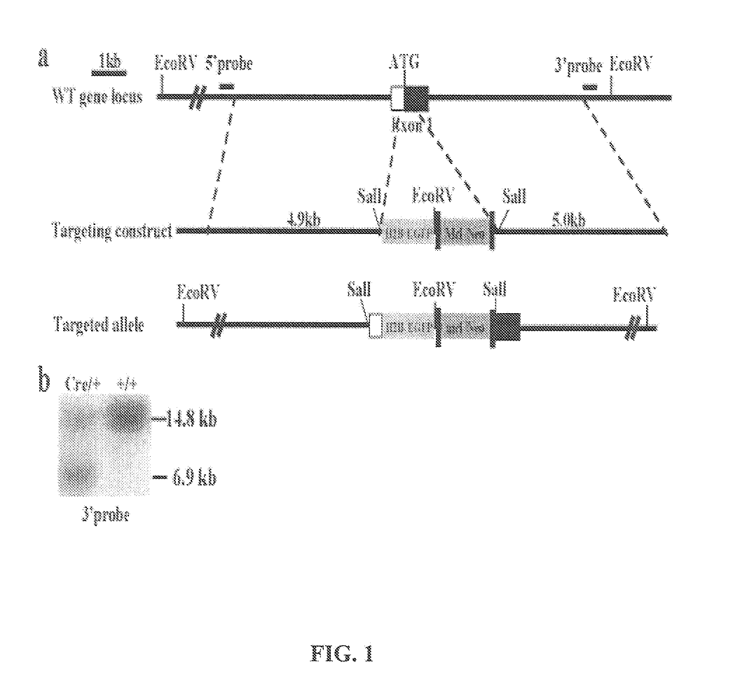

Generation of HCN4 nLacZ/H2B-EGFP and Cre knock-in mice—To better understand the ontogenesis of cardiac pacemaker and conduction cells and trace HCN4 cells during normal or abnormal heart and conduction system development, several HCN4 knock-in mouse lines were generated. The targeting vectors were designed as shown in FIG. 1A. A 4.9 kb 5′ arm and a 5 kb 3′ arm were generated by PCR amplification using SV129 mouse genomic DNA as template. The targeting cassette containing nuclear LacZ or histone 2B fused EGFP (H2B-EGFP) or CreET2 followed by FRT-mclNeo gene was introduced by homologous recombination into HCN4 locus before the HCN4 translation initiation site (ATG). Neomycin-resistant ES clones were screened for homologous recombination by Southern blot analysis. Two recombinant ES cell clones were used for the blastocyst injection and gave rise to germ line-transmitting chimeric mice that were crossed to Black Swiss female to generate heterozygous knock-in mice. Heterozygous knock-in mice are viable and are crossed to Black Swiss background.

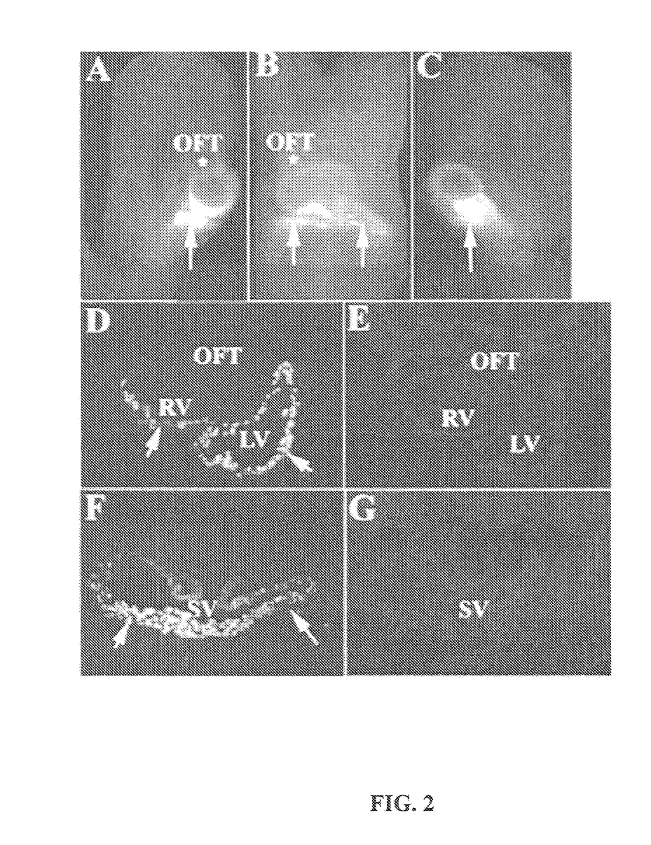

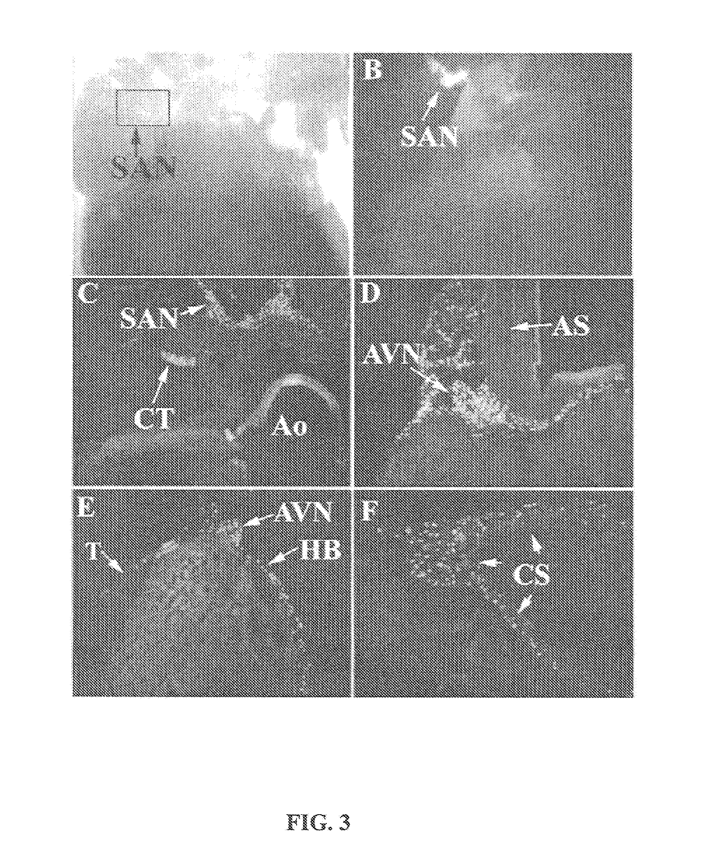

Characterization of HCN4-H2B-EGFP Expression—Analysis of EGFP expression in embryos at E8.5 and adult stages revealed a pattern of HCN4 expression that mimics the endogenous HCN4 mRNA expression as previously published (Moosmang, et al., Cellular expression and functional characterization of four hyperpolarization-activated pacemaker channels in cardiac and neuronal tissues. Eur J Biochem, 2001. 268(6): p. 1646-52; Shi, et al., Distribution and prevalence of hyperpolarization-activated cation channel (HCN) mRNA expression in cardiac tissues. Circ Res, 1999. 85(1): p. e1-6; and Garcia-Frigola, et al., Expression of the hyperpolarization-activated cyclic nucleotide-gated cation channel HCN4 during mouse heart development. Gene Expr Patterns, 2003. 3(6): p. 777-83). At E8-8.5 (FIG. 2), EGFP is strongly expressed in the region of cardiac inflow tract (FIGS. 2A to 2C and 2F arrow). Substantial EGFP expressing cells are also found in the presumptive left ventricle and relatively few in right ventricle (FIGS. 2A to 2E). Few if any EGFP cells are found in outflow tract (* FIGS. 2A, 2B and 2D). In adult, expression of HCN4 is confined to and delineates most components of the CCS, including sino-atrial node (SAN) (FIGS. 3A to 3C), and its extension-crista terminalis (CT) (FIG. 3C), atrioventricular node (AVN) and its extension (FIGS. 3D and 3E), and His-Purkinje fibers (FIG. 3E). HCN4 is also expressed in the retroaortic and atrioventricular ring (FIG. 3C, not shown), and on the right half of wall of the atrial septum (AS), which interconnects the SAN and AVN (FIG. 3D). HCN4-EGFP cells are also found in the coronary sinus (CS), in which pacemaker activity has been reported (FIG. 3F) (Moorman, et al., Development of the Cardiac Conduction System. Circ Res, 1998. 82(6): p. 629-644).

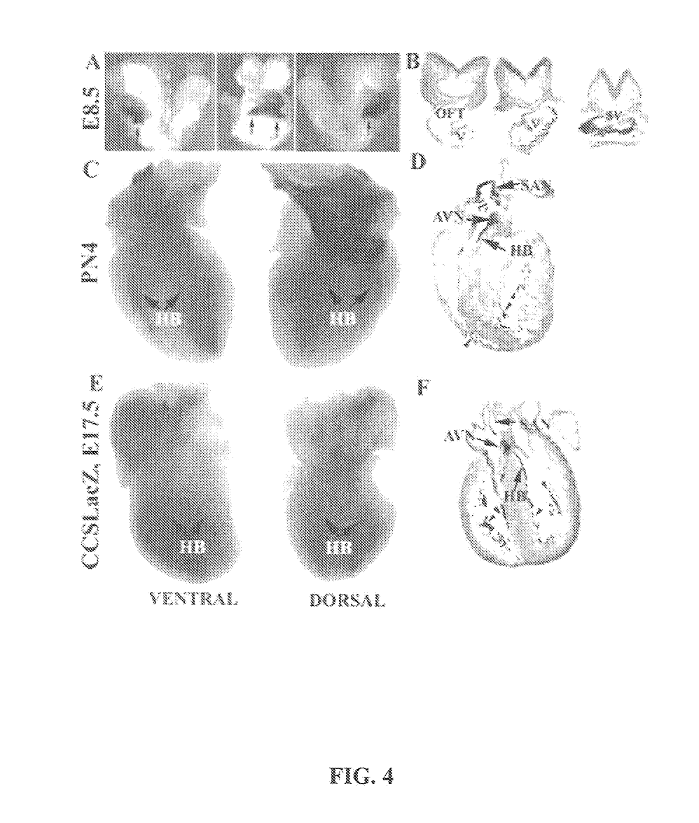

Characterization of HCN4-nLacZ expression in the cardiac conduction system —At E8-8.5, expression of HCN4-nuclear LacZ (nLacZ) is identical to that of HCN4-H2B-EGFP. HCN4-nLacZ is expressed in a posterior to anterior gradient, with strongest staining seen in the inflow tract (FIGS. 4A and 4B). Few if any LacZ cells are detected in the region of OFT (FIG. 4A middle panel, and 4B left panel). In postnatal heart, HCN4-nLacZ is detected in all components of the CCS, including SAN, AVN and His-Purkinje fibers (FIGS. 4C and 4D). In addition, the overall pattern of HCN4-nLacZ is similar to that of CCS-LacZ (FIGS. 4E and 4F), with the exception that HCN4-nLacZ is more strongly expressed in central components of the CCS whereas CCS-LacZ is more broadly expressed in His-Purkinje fibers (FIGS. 4D and 4E, right panel). However, the latter could due to the fact that HCN4-nLacZ is nuclear and CCS-LacZ is cytosolic. In addition, expression of HCN4-nLacZ in His-Purkinje fibers is more prominent in the left ventricle whereas expression of CCS-LacZ is equal in both right and left ventricles.

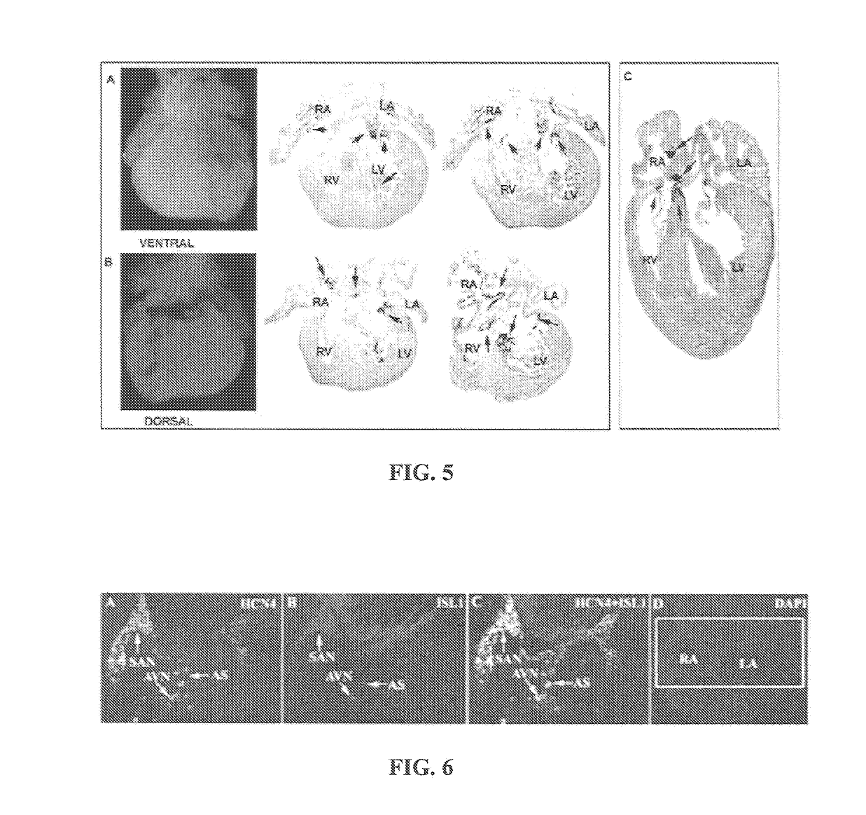

Lineage Analysis of HCN4 Expression in Heart—To better understand the contribution of HCN4 cells to the cardiac condution system during mouse heart development, HCN4 inducible Cre mice (HCN4-CreET2) were crossed to R26R-LacZ or β-actin-nLacZ indicator mice. Pregnant females were given a single intraperitoneal injection of tamoxifen (Sigma) at a dose of 50 mg/kg at desired time points (Sun, et al., Islet 1 is expressed in distinct cardiovascular lineages, including pacemaker and coronary vascular cells. Dev Biol, 2006). The embryos were harvested at distinct time points following injection and X-gal staining was performed. Analysis of hearts from mice induced at E7.5 and harvested at E 15.5 revealed expression of LacZ in myocardium, mostly in left ventricular trabeculae and right atria (FIGS. 5A and 5B). A few X-gal positive cells were also observed in the crest of the ventricular septum, left atria and right ventricle. Cells in the region of the SAN and AVN were also strongly marked (FIGS. 5A and 5B). Induction performed at postnatal day 1 and harvest at day 7 revealed expression of X-gal in all components of the CCS (FIG. 5C), a pattern that is essentially identical to that observed with HCN4-EGFP/nLacZ.

A subpopulation of HCN4 expressing cells co-expresses a marker of the second heart field—Emerging evidence has demonstrated that the formation of heart is accomplished by the migration and integration of cells from distinct origins and heart fields. Our lab has demonstrated that Islet1 (Isl1), a LIM-homeodomain (HD) transcription factor, is a marker for cardiac progenitors of the second heart field. Isl1⁺ cardiac progenitor cells contribute to various cardiovascular lineages, including cells in the CCS (Sun, et al., Islet 1 is expressed in distinct cardiovascular lineages, including pacemaker and coronary vascular cells. Dev Biol, 2006). As shown in FIG. 6, at E11.5, a subpopulation of HCN4 expressing cells in the region of the SAN and a few HCN4 expressing cells in AVN coexpress Isl1, suggesting, at least, a subset of cells in nodal tissues might come from the second heart field.

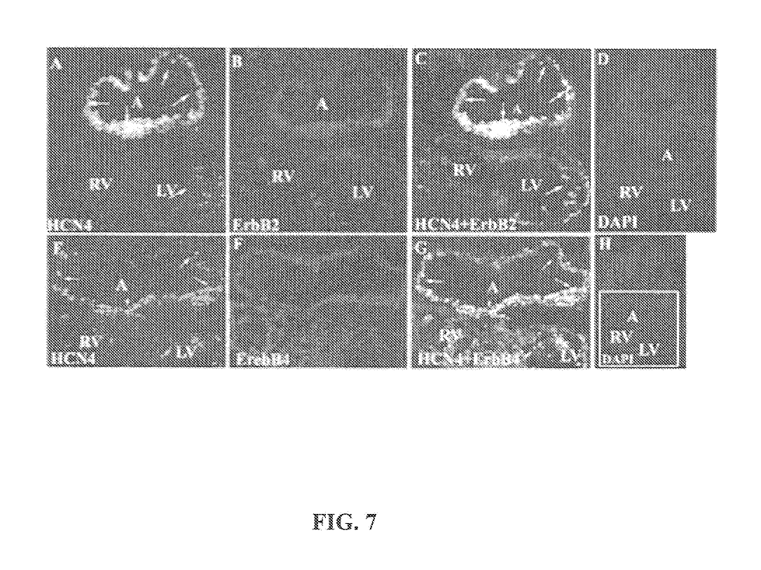

HCN4 is coexpressed with ErbB2 and ErbB4 in cardiomyocytes in both atria and ventricles—The receptor tyrosine kinases, ErbB4 and its coreceptor ErbB2, play an important role in heart development. Mutant mice defective in each of these receptors or their ligand, Neuregulin-1, die at around E10.5 due to trabecular defects, a substrate for the development of Purkinje fibers. As a first step to investigate a direct role of Nuregulin/ErbB signaling in the development of CCS by using HCN4-Cre, co-expression of HCN4 and ErbB was studied. As shown in FIG. 5, HCN4 is strongly co-expressed with ErbB2 (FIGS. 7A to 7D) as well as ErbB4 (FIGS. 7E to 7H), in the myocardium of both common atria and ventricles.

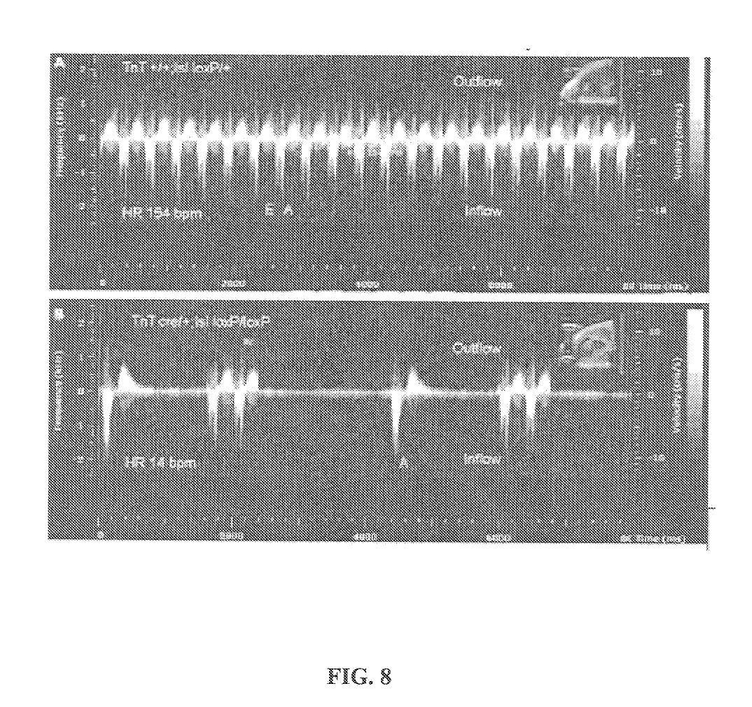

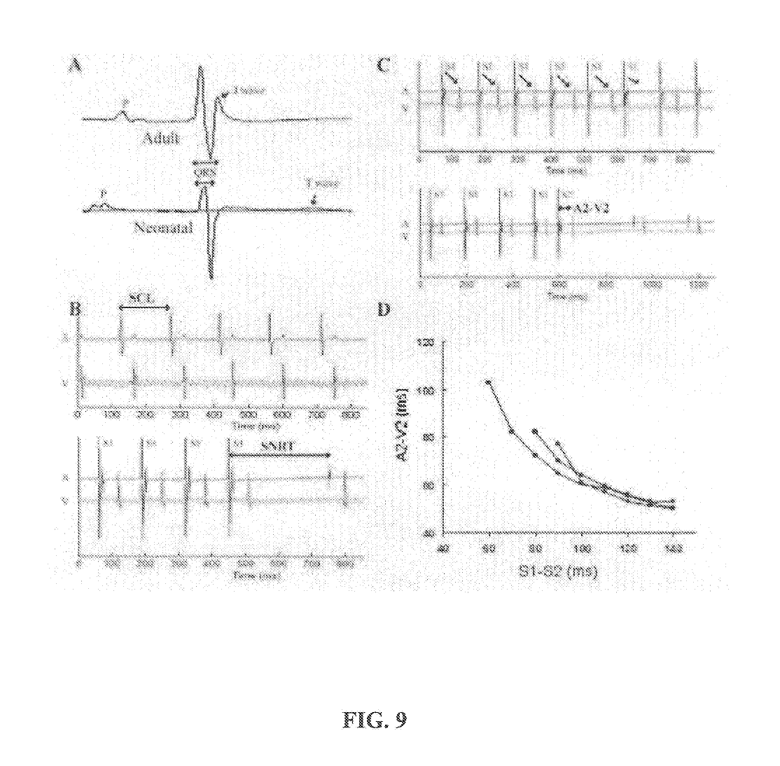

Cardiac electrophysiological and functional characterization in embryonic, fetal, neonatal, and adult mice—For functional characterization of embryonic, fetal, neonatal and adult mouse heart, a number of cardioelectrophysiological and functional analyses in experiment mice were performed at different stages, including high-quality telemetered electrograms that provided insight into the nature and origin of cardiac arrhythmias and echocardiography, using both an HP5500 (15 MHz probe) as well as the Vevo770 (FIG. 8). In addition, techniques were developed for monitoring conduction system function in fetal, neonatal and adult mice, either in whole animal or in isolated cardiac preparations (FIG. 9). Conditional HCN4 mutant and Isl1 mutant mice are being analyzed at distinct developmental stages, including embryonic stages.

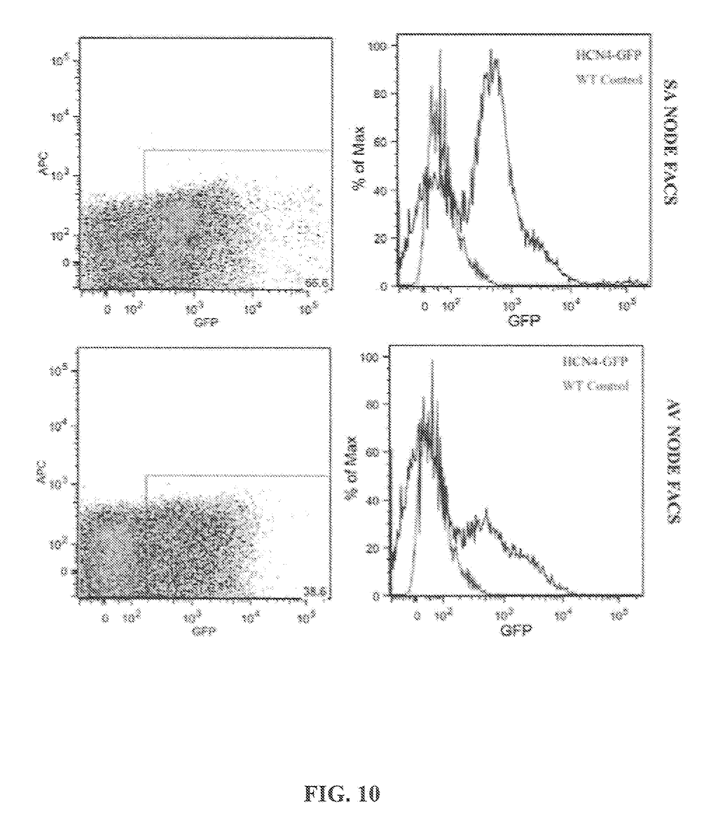

FACS Sorting of HCN4-EGFP Cells from Sinoatrial node and Atrioventricular Node—Hearts from 7-day-old HCN4-EGFP mice were dissected in Tyrode solution and examined under a fluorescent dissection microscope for the presence of EGFP in the SA nodal region. EGFP positive hearts were kept in Ringers buffer or DMEM with 10% FCS on ice for further dissection. Dissection of nodal tissues was guided by shuttling between fluorescent and bright illumination. Dissected EGFP nodal tissues were reconfirmed under the fluorescent microscope. Non-EGFP tissue was removed as this step could reduce total time required for FACS sorting. Nodal tissues were dissociated enzymatically and mechanically and the resulting single cell suspension was passed through a 40 μm filter. Quantification of FACS Data is shown in FIG. 10. From four EGFP SA nodal tissues, 11,131 EGFP negative and 1,220 EGFP positive cells were sorted and collected. From four AV nodal tissues, 47,756 EGFP negative and 4,428 EGFP positive cells were sorted and collected. The cells were kept in Trizol at −70° C. and total RNA was isolated. From 1,220 SA nodal cells, around 200 ng of total RNA was isolated.

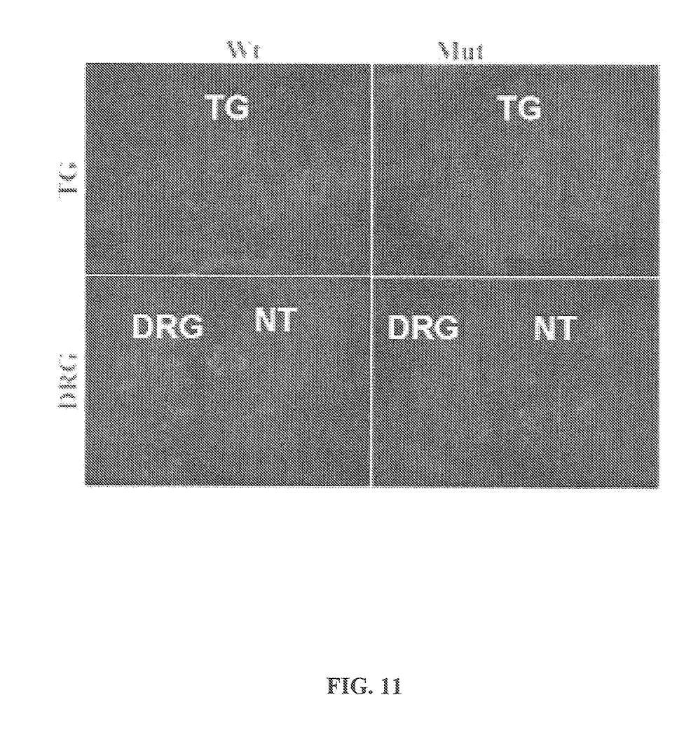

In previous microarray analysis of isolated dorsal root ganglia, a reproducible pattern of gene expression changes in Islet1 mutant mice was defined, which can be divided into up, down and ectopically expressed gene expression groups. Targets with the greatest fold changes, representing genes known to be critical in nervous system development, were verified by in situ and antibody staining. Results of analysis for one of the defined targets, Runx 1 are shown in FIG. 11.

### Example 2

### Ontogenesis of Pacemaker and Conduction System Cells

This examples provides the characterization of HCN4 lineages in heart by crossing HCN4 inducible Cre mouse line, with LacZ indicator (R26R1acZ or β-actin-nLacZ) mice. Investigation of the cardiac lineage origin of HCN4⁺ cells in the heart by using HCN4-EGFP mice, and those Cre mice which specifically mark these distinct cardiac lineages, such as Nkx2.5-Cre (primary and second heart field), Isl1-Cre (second heart field), and Tbx18-Cre (proepicardium).

Lineage Analysis—HCN4-CreET2 mice will be crossed with R26R-LacZ or β-actin-nLacZ indicator mice. The presence of a vaginal plug is designated as embryonic development day 0.5 (E0.5). The hearts from age or somite matched HCN4-nLacZ, H2B-EGFP and CCS-LacZ embryos or mice will be analyzed in parallel to that of HCN4-CreET2 lineage mice. To induce CreET2 activity, pregnant mice are given a single intraperitoneal injection of tamoxifen (Sigma) at a dose of 50 mg/kg at desired time points. To get reliable results, a minimal of 3-5 pregnant female mice will be needed at each time points. For earlier embryos (E6.5 to E8.5), low doses will be used to avoid tamoxifen toxicity. Embryos will be harvested at desired time points after injection and processed for X-gal staining or immunostaining.

Further characterization of HCN4 knock-in mice—The HCN4-nLacZ mice will be analyzed in parallel to CCS-LacZ mice, an existing CCS indicator mouse line. Wholemount and section X-gal staining at somite matched stages will be analyzed side by side, keeping in mind that these two mouse lines contain different β-galactosidase reporters, nuclear or cytosolic. Coimmunostaining of HCN4-EGFP with CCS-LacZ and connexins. HCN4-EGFP mice will be crossed with CCS-LacZ line and the hearts from resulting double heterozygous mice or embryos will be harvested at different stages and analyzed by immunostaining with anti-β-gal and visualization of GFP, as performed previously (Sun, et al., Islet1 is expressed in distinct cardiovascular lineages, including pacemaker and coronary vascular cells. Dev Biol, 2006). Colocalization of EGFP with connexins will be examined by direct visualization of GFP and immunostaining with anti-connexin antibodies: connexin 40, which is expressed preferentially in CCS and connexin 45, which is the only connexin expressed in early conduction system, and later expressed preferentially in the central conduction system. Investigation of the proliferative potential of HCN4 expressing cells in the early cardiac region by anti-phosphorylated histone H3 or BrdU labeling as described (Liang, et al., Pinch' Is Required for Normal Development of Cranial and Cardiac Neural Crest-Derived Structures. Circ Res, 2007).

Investigation of the cardiac lineage origin of HCN4⁺ cells in the heart—HCN4-EGFP mice will be first crossed on a homozygous R26R-LacZ or β-actin-nLacZ indicator background. Resulting HCN4-EGFP mice with LacZ indicator will be crossed with Cre mouse lines which specifically mark distinct cardiac lineages, such as Nkx2.5-Cre (primary and second heart field), Isll-Cre (second heart field), and Tbx18-Cre (proepicardium). R26R-LacZ or β-actin-nLacZ indicator will allow us to trace the Cre expressing cell lineages. Resulting embryos will be harvested at desired time points and colocalization will be examined by immunostaining of β-gal and direct visualization of EGFP or immunostaining with GFP antibody. To visualize active expression of Nkx2.5 and Isll in CCS, antibodies to Nkx2.5 and Isll will be used, and coimmunostaining will be performed on HCN4-EGFP samples.

Embryo dissection and X-gal staining and histology analysis—Pregnant females will be sacrificed at desired time points, and embryos will be dissected, examined, photographed. Embryos will be harvested and fixed for 0.5-3 hours in 4% paraformaldehyde (PFA). To optimize tissue fixation and penetrance of X-gal substrate (Roche Molecular, Indianapolis, Ind.), the chest wall will be opened before fixation and in some cases the heart will be dissected and incubated in substrate. Whole embryos or hearts will be incubated in X-gal solution (5 mM K₄Fe (CN)₆, 5 mM K₃Fe (CN)₆, 2 mM MgCl₂, 0.01% NP-40, 0.1% deoxycholate, 0.1% X-gal in PBS) at 37° C. For high-resolution analysis of β-gal activity, embryos were paraffin embedded, sectioned and counterstained with nuclear Fast Red.

Immunostaining—Dependent on the age of the samples, embryos or dissected hearts will be fixed for 0.5 to 3 hours in 4% PFA at 4° C., embedded in OCT, and cut to 5-10 μm sections. A significant loss of GFP intensity has not been observed by fixing the sample for less than one hour. The following antibodies will be utilized: Nkx2.5 (1:50, Santa Cruz, sc14033) that marks the primary and second heart field derived cells; isl1 that marks the progenitor cells from the second heart field (mouse monoclonal, 1:100, DSHB, or Rabbit polyclonal, 1:1000), anti-β-gal (goat, 1:200 Biogenesis, Rabbit, 1:1000, Cappel); Phosphorylated Histone H3 (upstate, 1:200). Sections will be incubated with fluorescently labeled antibodies after washing with 0.25% Triton X-100 in PBS. The specimens will be mounted with Vectashield DAPI medium (Vector Laboratory) and will be analyzed under a fluorescence microscope.

Detailed lineage analysis of HCN4 expressing cells combined with the analysis of HCN4-nLacZ/EGFP mice will allow direct visualization of CCS formation. Results of these studies will define the ontogenesis and lineage identities of HCN4 expressing cells in the heart and their respective contributions to the formation of distinct components of the CCS. Together, these studies will substantially advance the understanding of the ontogenesis, lineage differentiation and morphogenesis of CCS.

Lineage studies performed with the inducible HCN4-Cre mouse line will demonstrate whether cardiac cells that express HCN4 will go on to contribute only to conduction system lineages, or other cardiac lineages, for example working cardiomyocytes in which HCN4 expression is down regulated. HCN4-derived cells which do not contribute to the conduction system may be more susceptible to re-express HCN4 under conditions of stress or cardiac injury, and therefore may provide an arrhythmogenic substrate. In this regard, the current studies will provide significant mechanistic insights into human arrhythmogenesis and facilitate the development of effective intervention and regenerative therapies. In addition, the mouse lines characterized in this study represent valuable tools for multiple applications in the study of CCS.

### Example 3

### In Vivo Requirement for ErbB2 and/or ErbB4 Signaling in CCS Development

This example demonstrates that HCN4 marks cells with CCS potential that can be recruited in a temporally and spatially specific manner by ErbB2 and/or ErbB4 signaling to adopt a conduction system fate. CCS-specific deletion of ErbB2 and/or ErbB4 is achieved by crossing floxed ErbB2 and ErbB4 mice with HCN4-CreET2 mice and Cre activity will be induced by tamoxifen injection. Conduction system phenotypes will be investigated by histological, functional and electrophsiological analyses.

Generation of a mouse line in which ErbB2 or ErbB4 is specifically ablated in CCS—In the current preliminary studies, an HCN4-CreET2 knock-in mouse line was generated. Floxed ErbB2 and ErbB4 mouse lines were then obtained. Homozygous floxed alleles of ErbB2 and ErbB4 have been crossed on homozygous R26R-LacZ background to enable us to trace HCN4 cell lineages and monitor the HCN4-CreET2 efficiency. In a parallel experiment, foxed ErbB2 and ErbB4 mice have been crossed on a CCS-LacZ indicator background. Mice which are doubly heterozygous for HCN4-CreET2 and floxed ErbB2 or ErbB4 were generated. Crossing these doubly heterozygous mice to mice which are homozygous foxed ErbB2 or ErbB4 (on indicator backgrounds), mouse lines in which ErbB2 or ErbB4 is specifically ablated by HCN4-CreET2 in the presence of tamoxifen will then be generated. Tamoxifen injections will be given at E8 to E10.5 or later to determine temporal requirement of NRG/ErbB signaling for CCS formation, which may affect subpopulations of HCN4 expressing cells that contribute to distinct components of CCS. Cre efficiency will be monitored by the expression of LacZ indicator (R26R-LacZ), Western blot or immunostaining of heart tissue for the expression of ErbB receptors. Preliminary experiments will determine if ablation of ErbB2 or ErbB4 cause embryonic lethality and when mutants would die. If the mutants die embryonically, the timing of lethality will be determined, and embryonic cardiac function will be assessed in mice at least one day prior to their death.

Embryo dissection and X-gal staining and histology analysis—If the mutant mice die embryonically, the embryos or hearts will be harvested at least one day prior to their death and processed to X-gal staining and histological analysis. If the mutants survive to postnatal or adult stages, hearts of the mutant and control mice at postnatal day 1 (P1), one month, three month and 6 month will be harvested and analyzed by X-gal staining and histology. Morphology of trabaculae and quantification and topographical distribution of lineage labeled cells in the heart will be analyzed carefully.

Cardiac electrophysiological and functional characterization in embryonic, fetal, neonatal, and adult mice—For functional and physiological assessments, a minimum of 10 mice in each of mutant and control group will be needed, depending on the consistency of phenotypes observed. If mutant mice die embryonically, mouse echocardiography will be utilized. The Visualsonics Vevo770 high-resolution ultrasound system will be used for ultrasound biomicroscopy (UBM) to identify physiologic data in utero. Using the RMV704 probe (40 MHz), embryos from E9-14 can be visualized and identified for phenotype-genotype correlation. This imaging technique allows embryos to remain in their normal in utero environment at physiological temperatures and yields reliable physiological data. For each embryo, B-mode, M-mode, and pulse wave Doppler images can be obtained to allow for assessment of ventricular function, heart rate, and estimate of stroke volume.

If mutant mice survive to postnatal or adult stages, mobility and survival of the mice will be monitored and electrophysiological and functional analyses will be performed periodically (once every two weeks or more). Depending on the phenotypes observed, telemetery recordings of the electrocardiogram can be performed to monitor cardiac rhythm and rate in non-anaesthetized animals. If the mutant mice developed cardiac arrhythmias, optical mapping will be performed as will intracardiac electrograms that provide insight into the nature and origin of cardiac arrhythmias. Echocardiography will be examined to assess cardiac morphology and function. To examine the function of SAN and AVN, more detailed electrophysiological assessments can be done on isolated hearts from neonatal and adult mice as described in preliminary studies.

In situ hybridization and immunostaining—A number of distinct markers that mark the distinct components of CCS during development and/or adults will be analyzed by in situ hybridization or immunostaining if antibody available. Those include Islet1, Nkx2.5, GATA-4, -6, Tbx3, Tbx2, Tbx5, HF-1b, Msx-2 and connexins. Target genes from microarray analysis will be analyzed on ErbB mutant. In vitro analysis will also address whether the recruitment of CCS cells by neuregulin is ErbB receptor dependent.

Generation of a mouse line in which ErbB2 and ErbB4 is specifically ablated in CCS—Doubly heterogygous HCN4-CreET2 and ErbB2 or ErbB4 mice will be crossed with homozygous ErbB2 and ErbB4 mice with R26R-LacZ or CCS-LacZ indicator background as described above. Pregnant female at E7.5-8 will be injected with tamoxifen. Embryos will be harvested 48 hours after injection (at E9.5-10).

Whole embryo culture or organ culture—Culture of whole embryos or dissected hearts will be performed. Briefly, E9.5-10 embryos or hearts will be dissected in PBS. Whole embryo or hearts will be cultured in 12-well plate in DMEM with 10% FBS and 50 mM HEPES and 1% Pen-Strep, in the presence (+) or absence (−) of 2.5 nM NRG-1 (R&D System), at 37° C., 5% CO₂ in culture incubator. Initial heart rates will be counted 4 hours after culture and then heart rates will be measured everyday. Yolk sac will be genotyped to identify mutant embryos or hearts (HCN4-CreET2+/−; ErbB2/4f/f; R26R-LacZ+/− or CCS-LacZ+/−) and controls embryos or hearts (HCN4-CreET2+/−; R26R-LacZ or CCS-LacZ+/−). At the end, the samples will be fixed and subjected to analysis by X-gal staining as described above.

Analysis by X-gal staining—X-gal staining will be performed as described above. By comparing CCS-LacZ expression patterns in hearts of HCN4-CreET2; ErbB mutants and the control embryos cultured in the presence of NRG-1, direct evidence will be provided as to whether the recruitment of cells to CCS lineage by ErbB receptors is NRG-1 dependent and whether response to NRG-1 will be abolished in the absence of ErbB receptors. Cultures without added NRG-1 represent the response to endogenous NRG-1. By comparing R26R-LacZ expression pattern of hearts from HCN4-CreET2;ErbB mutants and control embryos cultured with or without NRG-1 added, it will be determined whether ErbB signaling pathway is required for the recruitment of HCN4 expressing cells to CCS and whether this NRG effect will be abolished in the absence of ErbB receptors. Cultures without added NRG-1 represent the response to endogenous NRG-1. Data will be presented as mean ±SD and analyzed by the t test or ANOVA when comparing multiple groups. P≦0.05 is considered statistically significant.

Results of these studies will address the in vivo requirement for ErbB2/ErbB4 signaling in the development of CCS. Although previous studies have demonstrated that application of NRG-1 can recruit cells to CCS lineages, whether this signaling pathway is required in vivo has not been addressed. It is anticipated that ablation of either ErbB2 or ErbB4 receptors alone in HCN4 expressing cells will result in significant and adverse alterations in conduction system formation and/or function. Alternatively, ablation of ErbB2, but not ErbB4, may result in a conduction system phenotype. This may indicate that another partner of ErbB2, for example ErbB3, may be involved in conduction system formation. In this event, while unlikely, given previous studies and the preliminary results provided herein demonstrating co-localization of HCN4 and ErbB receptors, this possibility would be investigated by first examining expression of ErbB3 and HCN4, and if warranted, examining ErbB3 mutants for a potential cardiac conduction phenotype. In any case, results of these studies will demonstrate whether ErbB2/4 signaling is required for conduction system formation. If conduction phenotypes are observed in HCN4-CreET;ErbB mutants, characterization of the phenotype will give insights as to the specific manner in which this signaling is required for CCS formation and function.

By ablation of ErbB signaling in a temporal manner, the studies provided herein will additionally address requirements for ErbB signaling through specific ErbB receptors in formation and function of distinct components of the conduction system, including the His-Purkinje system and nodal tissues. It may be found that the fast-conducting tissues are preferentially dependent on ErbB signaling, or alternatively, that all conduction system components are dependent on this pathway. Furthermore, by performing in vitro explant studies of hearts from HCN4-CreET2;ErbB mutants and littermate controls, it will be established whether previously observed effects of neuregulin are mediated by ErbB receptors.

The deletion efficiency of HNC4-CreET2 in ablating ErbB receptors, in concert with the stability of preexisting ErbB receptor protein, may affect the observed data, as previous in vitro studies have demonstrated that responsiveness to neuregulin signaling in CCS development is confined to a short developmental stage before E9.5. It is therefore proposed to inject tamoxifen relatively early (around E8) to allow time for degradation of existing ErbB receptors, but injection at the earliest times that HCN4 is expressed may be required. To potentially ameliorate this issue, one null allele of ErbB (ErbB2 or 4+/−) could also be generated by using protamine Cre mice. Crossing this mouse line to HCN4-CreET2 mice will generate lines which are HCN4-Cre+/−;ErbB2 or 4+/−. These lines could each be crossed to homozygous floxed ErbB2 or 4 to generate CCS-specific ErbB knockout mutant.

If no conduction system phenotype is observed, one possibility is that ErbB2 and ErbB4 do not play a significant role on the conduction system. However, this is highly unlikely, given previous evidence and the preliminary results provided herein that show coexpression of HCN4 with these receptors. Another possibility is that the previously observed requirement for NRG-1 signaling in conduction system induction could be an indirect, or cell nonautonomous effect, although this is unlikely given co-immunostaining of ErbB receptors with HCN4-EGFP. If there is no phenotype, ErbB2 and ErbB4 will be ablated with other Cre mouse lines expressed in other tissues present in the developing heart during the pertinent time window. Expression of the ErbB receptors will be examined in these tissues, including the endocardium and epicardium. These include abation with Tie2-Cre within endothelial lineages, including the endocardium, and Tbx18-Cre within epicardial lineages which are present from E9.5. A more pan-cardiomyocyte specific Cre, cTnT-Cre would also be utilized. MLC2v-Cre mediated ablation of ErbB2 has been reported. Mutant mice survive to adult and develop dilated cardiomyopathy, but heart rate is normal. However, Cre efficiency of MLC2v-Cre is low, especially in embryonic stages. In MLC2v-Cre;ErbB2 mutant mice, ErbB2 protein levels were not reduced at El 8.5, suggesting inefficient ablation of ErbB2 with MLC2vCre mice. Therefore, from this study, the potential role of earlier NRG/ErbB signaling in CCS formation was not addressed. cTnTCre, mediates efficient excision in differentiated cardiomyocytes by E7.5, and is expressed throughout all myocardial chambers. It is anticipated that cTnT-Cre;ErbB mutant phenotypes would involve both cardiomyocytes and CCS.

As the HCN4-CreET2 provided herein is a heterozygous loss of function mutant, it is possible that this may affect the conduction system phenotype. Although this unlikely, as in the initial characterization of these mice, no physiological or electrophysiological phenotype has been detected.

### Example 4

### Genes Differentially Expressed

### In Nodal Cells of CCS and Non-CCS Cardiomyocytes

This example provides genes that are specifically or predominantly expressed in the sinoatrial or atrioventricular nodes may play important roles in the development, maintenance, and function of the CCS. GFP expressing nodal cells from HCN4-EGFP mice will be FACS sorted and utilized for microarray analyses, comparing to similarly purified non-CCS cardiomyocytes.

Cardiac pacemaker and conduction cells—Cells of SAN and AVN will be isolated as described above. Briefly, hearts from postnatal day 1 mice will be removed and selected under dissection microscope equipped with UV light for the presence of HCN4-EGFP in SAN. The positive hearts are pooled in oxygenated Tyrode solution (140 mM NaCl, 5.4 mM KCl, 1.8 mM CaCl₂, 1 mM MgCl₂; 10 mM glucose; 5 mM Hepes, pH 7.4) for further dissection. For SAN dissection, the right atrium will be cut from the heart and a thin strip of SAN tissue together the crista terminalis and the orifices of the venae cavae will be cut from the right atrium. Precise dissection is based on the anatomical criteria under the bright field and the presence of EGFP viewed under UV light with green fluorescent filter. In the preliminary studies provided herein, it was observed that substantial active expression of HCN4-EGFP occurred in crista terminalis (CT) in continuation with SAN. Therefore, CT is considered as the extension of SAN and is included in the SAN preparations. AVN and its extensions will be dissected under the fluorescent field. Dissected SAN and AVN tissues will be pooled separately and digested for 20-30 min in digestion buffer that contain 230 units/ml Type I collagenase; 15 units/ml Type IIA elastase in Ca²⁺-free Tyrode solution. The tissue will be triturated gently and let the cell clump set down and supernatants will be collected to a new tube. The cells clump will be digested for an additional 5 min and triturated. Cell suspensions will be pooled, washed and resuspended in DMEM with 10% FCS. To avoid small cell clumps that will give rise to false positive signals in FACS sorting, the cells suspension will be passed through a 40 μm filter. Non-EGFP cardiac tissues will be treated in a similar way and non-labelled cells will used to set the background fluorescence.

Control cardiomyocytes—Heart tissue is comprised of multiple cell populations, such as cardiomyocytes, fibroblasts, endocardial, endothelial and smooth muscle cells and the CCS cells. Careful consideration will be given to ensuring the purity of control cardiomyocytes devoid of other cell types. CCS cells express many cardiomyocyte-specific markers, such as α- and β-MHC. Taking this into account, αMHC-Cre mice will be crossed with Z/RFP mice, a RFP indicator mouse line. Resulting doubly heterozygous mice will be crossed with HCN4-EGFP mice to get triply heterozygous mice with αMHC -Cre+/−; Z/RFP+/−;HCN4+/− (named red control mice). The hearts from red control mice will be harvested and the tissues surrounding the SAN and AVN or ventricular septum will be dissected and treated as described above. The cells will be sorted into three fractions: 1) Red only, which represents pure cardiomyocytes. This fraction will be used as control; 2) Red and Green, which represents CCS cells with variable expression level of Red and Green; and 3) Non-fluorescent non-myocytes, including diverse cell types.

Total RNA isolation—Total RNA will be isolated with Ambion Message Amp II kit. The total amount of starting RNA for the previous experiments on dissected dorsal root ganglia was approximately 500 ng. Based on preliminary studies with HCN4-EGFP FACS sorted cells, it is estimated that around 40 to 50 neonates (from about 10 litters) will be required to provide adequate tissue to yield sufficient amounts of RNA. For control cardiomyocytes, 4-10 neonates will be needed, depending on the dissection area. A control mouse line will be established and preliminary experiments will be performed to ensure the purity of sorted EGFP and RFP cells by inspection under the fluorescent microscope, and RT-PCR analysis performed on sample RNAs from each cell population to examine the expression pattern of genes known to be differentially expressed in CCS or cardiomyocytes.

Affymetix array—The Affymetrix Mouse 430 expression array will be used. Genes that are differentially expressed in the sinoatrial node, atrioventricular node and/or cardiomyocytes will be validated by performing quantitative RT-PCR on RNA extracted from FACS sorted nodal cells and control cardiomyocytes. Potential targets will be validated by RNA in situ hybridization on whole mount or cardiac sections, depending on stages being examined, or by antibody staining of HCN4-EGFP heart tissue in cases where antibodies are available. Priority will be given to: 1) those genes with most significant fold changes; 2) those genes with known function but an unknown role in CCS; and 3) those genes with unknown function. Focus will be on the signaling pathways that potentially regulate the differentiation and maintenance of CCS functions.

This example will provide valuable information about the genes specifically or differentially expressed in SAN, AVN and cardiomyocytes, and will further the understanding of the gene regulation and function in nodal cells. The results will be interpreted in the context of the results of Example 2, the lineage origins of the SAN, or AVN. Distinct or similar embryonic lineage origins may be reflected in the constellation of genes observed to be specifically expressed within the SAN or AVN. Results of these studies will elucidate new genetic pathways required for conduction system maintenance and potentially for conduction system formation. Targets of interest will be investigated by in vivo loss of function experiments.

Although the SAN and AVN represent a relatively small population of cells, the preliminary results provided herein demonstrate the feasibility of obtaining sufficient numbers of purified cell populations to obtain adequate amounts of RNA for the proposed studies. However, changes in gene expression may occur during the in vitro isolation. It is likely that these studies will yield some genes which are differentially expressed between nodal cell populations and working cardiomyocytes. Additionally, all potential genes of interest will undergo in vivo validation, as described. Furthermore, given the believed close lineage relationship of nodal cells and adjacent cardiomyocytes, fold changes in gene expression might not be dramatic.

While genes identified from neonatal heart may or may not represent genes expressed at initial stages of CCS development, they are likely to represent pathways required for maintenance and function of conduction system, and therefore likely to be of critical importance in human disease. Additionally, it is highly likely that genes from this stage will also play a role in conduction system formation and development, as has been shown for several other genes, including the transcription factors Nkx2.5 and Tbx5.

Understanding of genes and signaling pathways underlying development of CCS is important to get molecular insights into human cardiac arrhythmia and eventually develop more effective intervention and regenerative therapies. Gene expression profiling of CCS has been hampered by limited cell numbers and the difficulty of distinguishing CCS cells from cardiomyocytes based on morphology in order to isolate highly purified conduction system cells for high-throughput gene analysis. In this respect, the HCN4-EGFP knock-in mouse provides us with an ideal model system to fulfill this goal. In previous studies, microarray analysis was successfully performed on small amounts of tissue from isolated dorsal root ganglia. In preliminary studies provided herein, HCN4-EGFP cells and non-EGFP cells were successfully sorted from neonatal SA and AV nodal regions. Together, these data demonstrate the feasibility of using HCN4-EGFP mice to analyze specific gene expression profiles in nodal cells of the CCS.

Genes identified based on the microarray analysis provided herein will be analyzed according to their selective expression within the sinoatrial node alone, the atrioventricular node alone, or expressed in common within both nodes. Priority will be given to genes with the greatest fold difference in expression between SA node, AV node, and non CCS-cardiomyocytes. Another selective criterion will be genes of known function which do not have a known role in CCS formation, and therefore may give us novel insights into conduction system maintenance or formation. Validation will come from examination of identified genes and comparison to genes known in the literature to be specifically expressed in conduction system tissue. Genes identified by microarray will be validated by quantitative RT-PCR performed on FACS sorted HCN4-EGFP expressing cells from each nodal area and control non-CCS cardiomyocytes, and RNA in situ hybridization analysis performed on tissue sections from neonatal heart. Identified genes will also be examined for their potential links to common biochemical pathways.

Although the invention has been described with reference to the above examples, it will be understood that modifications and variations are encompassed within the spirit and scope of the invention. Accordingly, the invention is limited only by the following claims.

1. An isolated population of cardiac conduction system (CCS) progenitor cells, wherein at least 90% of the cells express HCN4.

2. The isolated population of claim 1, wherein at least 90% of the cells co-express HCN4 and Islet1, HCN4 and Nkx2.5, or HCN4 and Tbx18.

3. (canceled)

4. A method of isolating CCS progenitor cells, the method comprising isolating cells that express HCN4 from a population of stem and/or progenitor cells.

5. The method of claim 4, comprising isolating cells that express HCN4 and an additional marker selected from the group consisting of Islet1, Nkx2.5, Tbx18, flk1, and LeX/SSEA1/CD15 from a population of stem and/or progenitor cells.

6. The method of claim 4, wherein the isolating comprises sorting the population of stem and/or progenitor cells based upon HCN4 expression levels.

7. The method of claim 4, wherein the isolating comprises sorting the population of stem and/or progenitor cells based upon the expression levels of a stem cell marker, and wherein the stem cell marker is selected from the group consisting of Islet1, Nkx2.5, Tbx18, flk1, and LeX/SSEA1/CD15.

8. The method of claim 4, wherein the population of stem and/or progenitor cells is obtained from heart tissue, epicardial tissue, serosal mesothelial tissue, adipose tissue, dermal tissue, bone marrow, umbilical cord, or embryonic tissue.

9. The method of claim 4, wherein the population of cells is a population of mouse or human cells.

10. The method of claim 4, wherein the CCS progenitor cells are capable of differentiating into sinoatrial node cells, atrialventricular node cells, and/or His-Purkinje fiber cells.

11. The method of claim 4, further comprising growing the population of stem and/or progenitor cells on feeder cells or conditioned medium or extracellular matrix derived from the feeder cells.

12. The method of claim 11, wherein the feeder cells comprise cardiac fibroblasts.

13. The method of claim 4, further comprising contacting the population of stem and/or progenitor cells with an agent that promotes or maintains the self-renewal capacity and/or suppresses differentiation of the population of stem and/or progenitor cells.

14. A method of isolating sinoatrial node cells or progenitors thereof, the method comprising:
contacting a population of stem and/or progenitor cells with an agent that promotes Islet1 expression; and
isolating cells that express HCN4 from the population.

15. The method of claim 14, wherein the population of stem and/or progenitor cells is obtained from heart tissue, epicardial tissue, serosal mesothelial tissue, adipose tissue, dermal tissue, bone marrow, umbilical cord, or embryonic tissue.

16. The method of claim 14, wherein the agent that promotes Islet1 expression is selected from the group consisting of a layer of feeder cells comprising cardiac fibroblasts, extracellular matrix or conditioned medium produced by the feeder cells, an FGF signaling agonist, a Wnt signaling agonist, a GSK-3beta inhibitor, an TGF-β/activin signaling agonist, a Smad1 antagonist, a Smad5 antagonist, a Smad8 antagonist, and any combination thereof.

17. The method of claim 14, wherein the isolating comprises sorting the population of stem and/or progenitor cells based upon HCN4 expression levels.

18. The method of claim 14, wherein the isolating comprises sorting the population of stem and/or progenitor cells based upon Islet1 expression levels.

19. The method of claim 14, wherein the sinoatrial node cells or progenitors thereof coexpress HCN4 and Islet1.

20-40. (canceled)
